(19) United States
(12) Patent Application Publication
(10) Pub. No.: US 2025/0197890 A1
(43) Pub. Date: Jun. 19, 2025

(54) TARGET CELL PREPARATION METHOD AND SYSTEM

(71) Applicant: KABUSHIKI KAISHA TOSHIBA, Tokyo (JP)

(72) Inventors: Shu OKUMURA, Oyama (JP); Mitsuko ISHIHARA, Setagaya (JP)

(73) Assignee: KABUSHIKI KAISHA TOSHIBA, Tokyo (JP)

(21) Appl. No.: 18/795,703

(22) Filed: Aug. 6, 2024

(30) Foreign Application Priority Data
Dec. 19, 2023 (JP) 2023-214003

(51) Int. Cl. C12N 15/88; B01L 3/00; C12N 13/00

(52) U.S. Cl. CPC C12N 15/88; B01L 3/502715; C12N 13/00; B01L 2200/16; B01L 2300/0861; B01L 2300/18

(57) ABSTRACT

According to one embodiment, a target cell preparation method includes forming a first water-in-oil droplet containing source cells by mixing an aqueous solution containing source cells with an oil phase, forming a second water-in-oil droplet containing liposomes by mixing a liposomal aqueous solution encapsulating an active agent with an oil phase, fusing the formed first water-in-oil droplet with the second water-in-oil droplet, and introducing the active agent into the source cells in the fused droplet.

### CROSS-REFERENCE TO RELATED APPLICATIONS

This application is based upon and claims the benefit of priority from Japanese Patent Application No. 2023-214003, filed Dec. 19, 2023, the entire contents of which are incorporated herein by reference.

### FIELD

Embodiments described herein relate generally to a target cell preparation method and system.

### BACKGROUND

In the field of regenerative medicine where cells are produced by gene transfer, it is desired to produce cells with high uniformity. For example, in the case of producing iPS cells by introducing the Yamanaka factor, which is an active agent, into cells, a number of types of genes need to be introduced into source cells. However, When generating cells by culturing the source cells on a petri dish and applying the active agent, the non-uniform contact between each cell and the active agent can result in a heterogeneous condition of the cells. Thus, there is a need for a method that ensures the active agent is introduced into each cell in a consistent amount to obtain cells of uniform quality.

### BRIEF DESCRIPTION OF DRAWINGS

FIG. 1 is a schematic diagram showing a first embodiment.

FIG. 2 is a schematic diagram showing the first embodiment.

FIG. 3 is an image diagram showing the first embodiment.

FIG. 4 is a scheme diagram showing a third embodiment.

FIG. 5 is an image diagram showing third, fifth, and sixth embodiments.

FIG. 6 is an image diagram showing a seventh embodiment.

FIG. 7 is an image diagram showing an eighth embodiment.

FIG. 8 is a schematic diagram showing a microfluidic system used in Example 1.

FIG. 9 is an image diagram showing results of Example 1.

FIG. 10 is an image diagram showing the results of Example 1.

FIG. 11 is a schematic diagram showing a microfluidic system used in Example 2.

FIG. 12 is a diagram showing results of Example 2.

FIG. 13 is a diagram showing results of Example 3.

FIG. 14 is a diagram showing results of Example 4.

### DETAILED DESCRIPTION

In general, according to one embodiment, a target cell preparation method comprises forming a first water-in-oil droplet containing source cells by mixing an aqueous solution containing source cells with an oil phase, forming a second water-in-oil droplet containing liposomes, which serve as carriers for an active agent delivery, by mixing a liposomal aqueous solution encapsulating an active agent with an oil phase, fusing the formed first water-in-oil droplet with the second water-in-oil droplet, and introducing the active agent into the source cells in the fused droplet.

Embodiments will be described hereinafter with reference to the accompanying drawings. In each embodiment, the same symbols are attached to substantially the same constituent parts, and their descriptions may be partially omitted. The drawings are merely schematic. The relationships between the thickness and the plane dimensions of each part, the ratios in thickness of the parts, and the like may differ from those in reality.

### First Embodiment

A first embodiment is a target cell preparation method. This method comprises forming a first water-in-oil droplet containing source cells by mixing an aqueous solution containing source cells with an oil phase, forming a second water-in-oil droplet containing liposomes by mixing a liposomal aqueous solution encapsulating an active agent with an oil phase, fusing the formed first water-in-oil droplet with the second water-in-oil droplet, and introducing the active agent into the source cells in the fused droplet. The method ensures that each droplet of have the same concentration of the active agent encapsulated within the liposomes, which allows for consistent interaction between source cells and agents in the fused droplet.

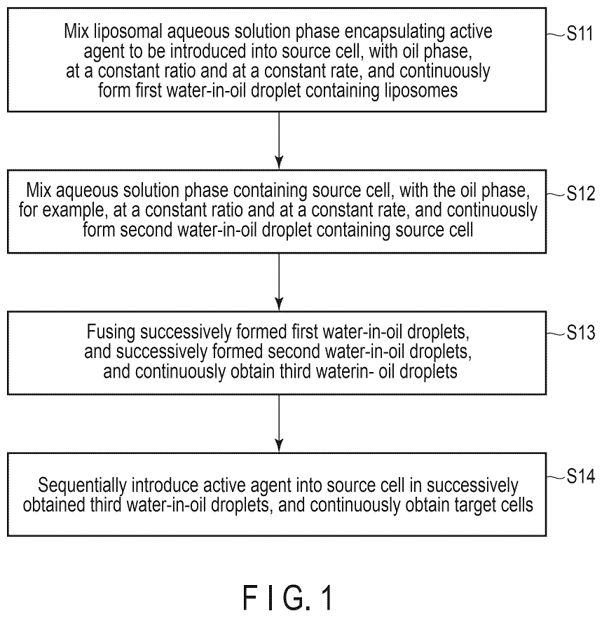

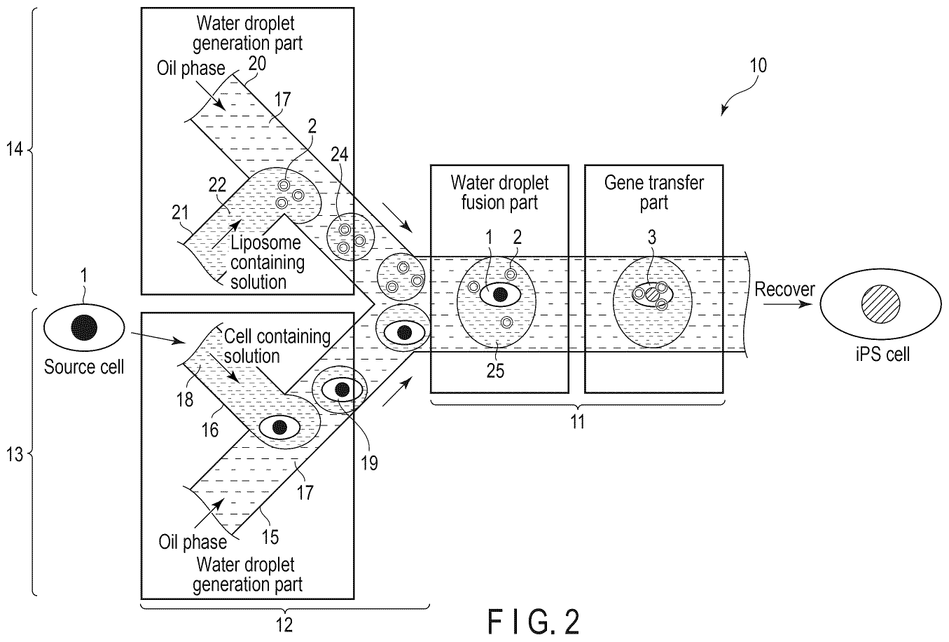

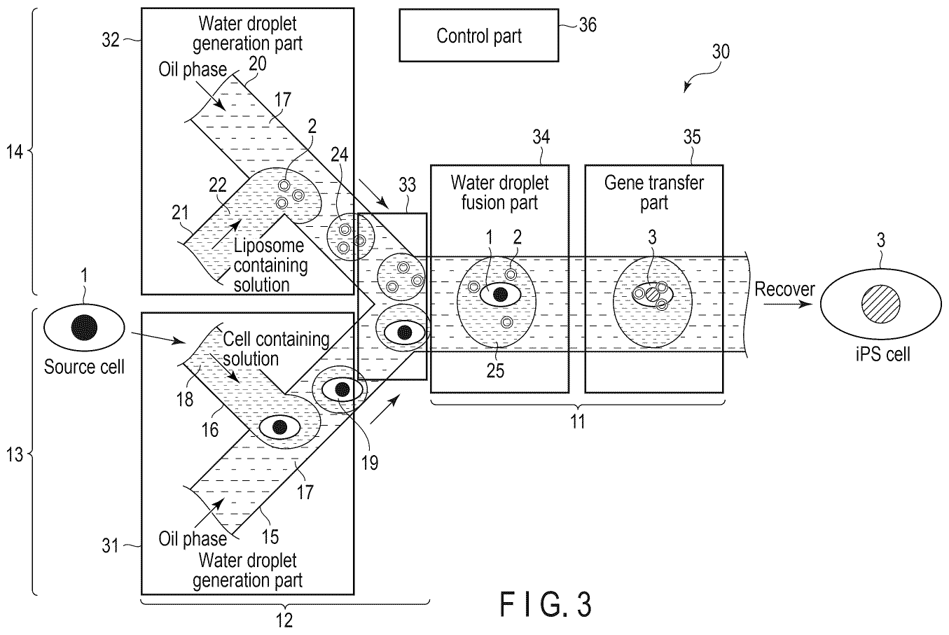

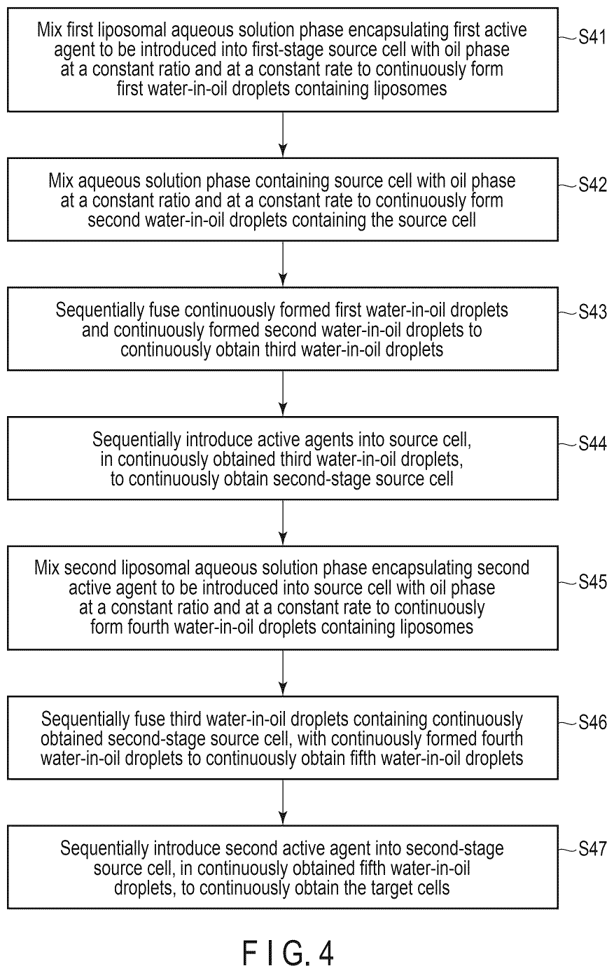

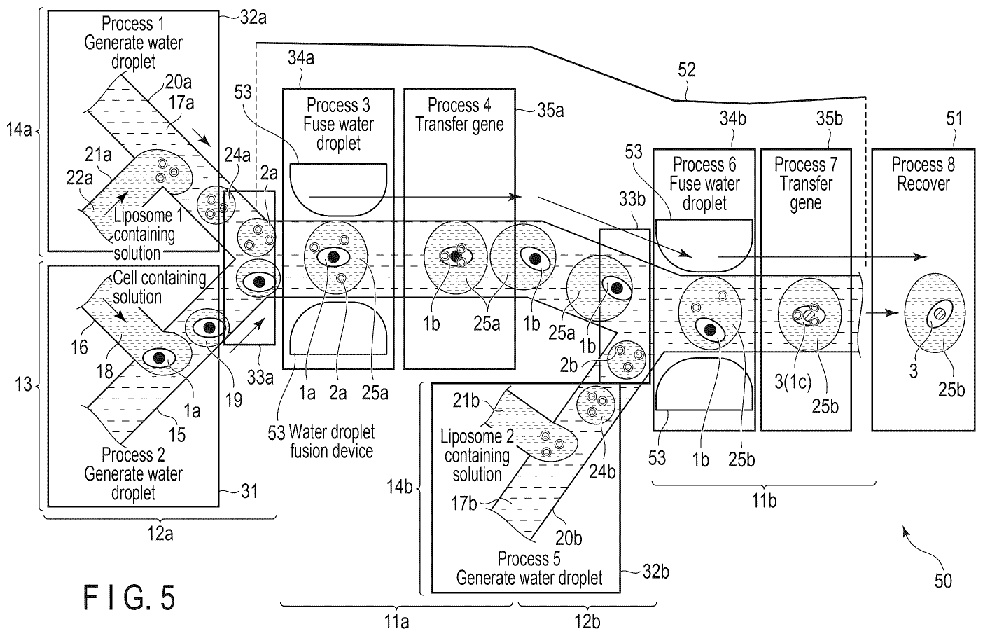

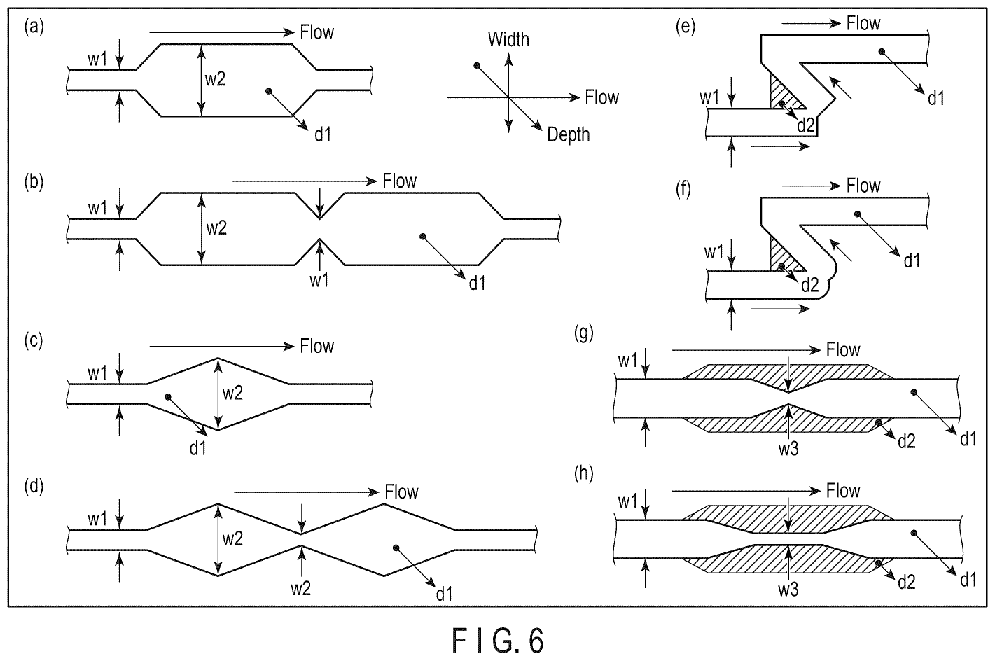

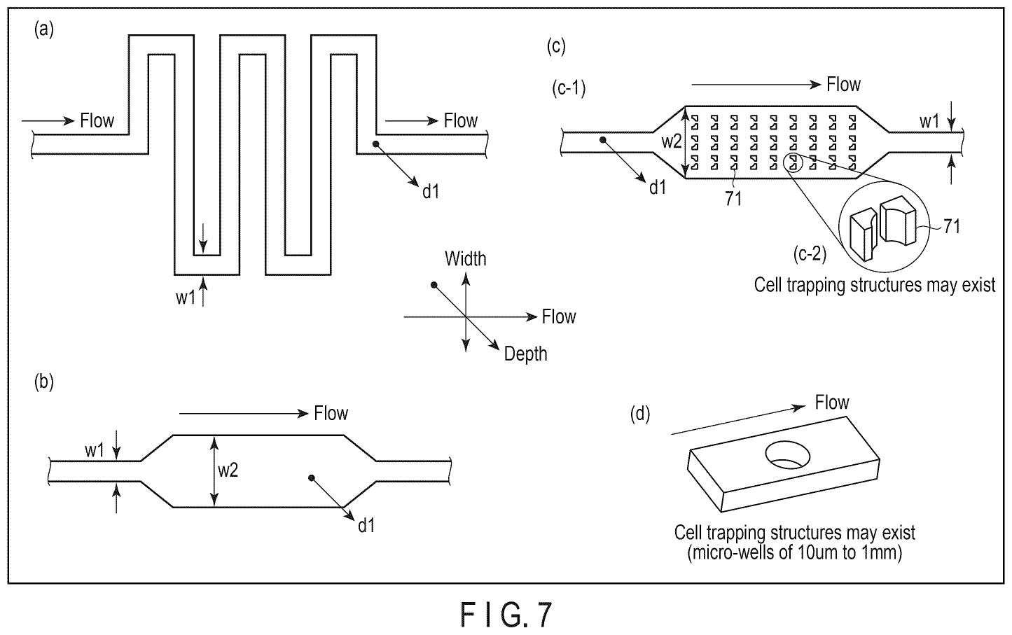

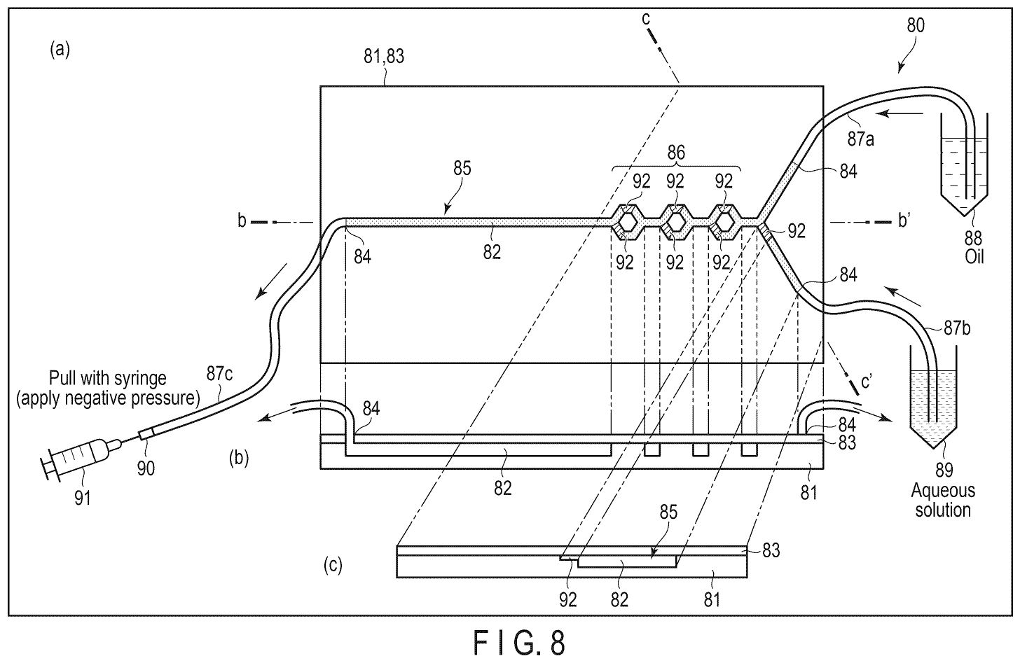

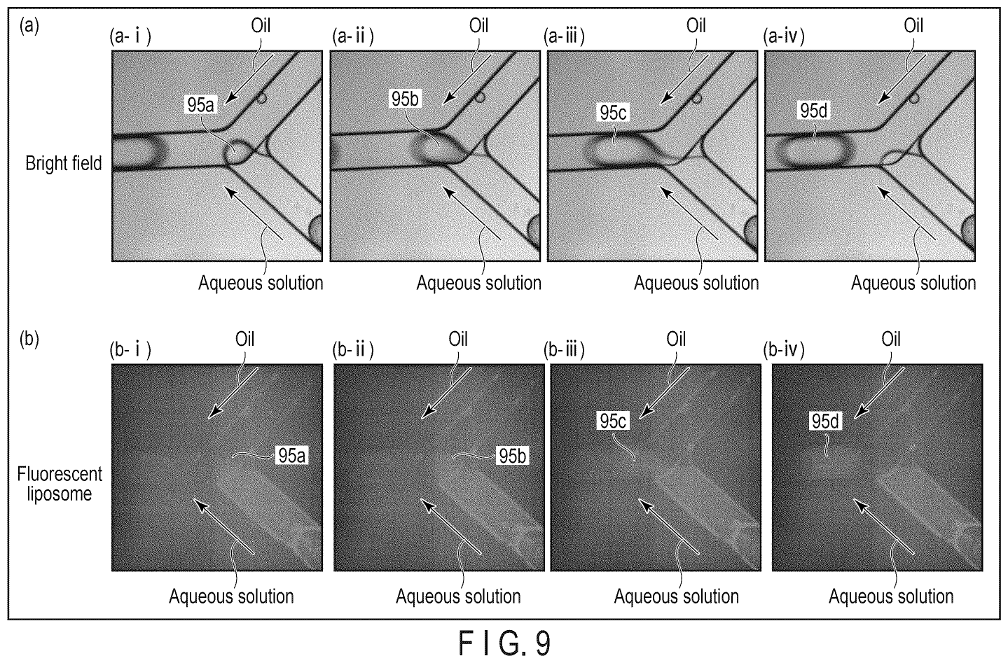

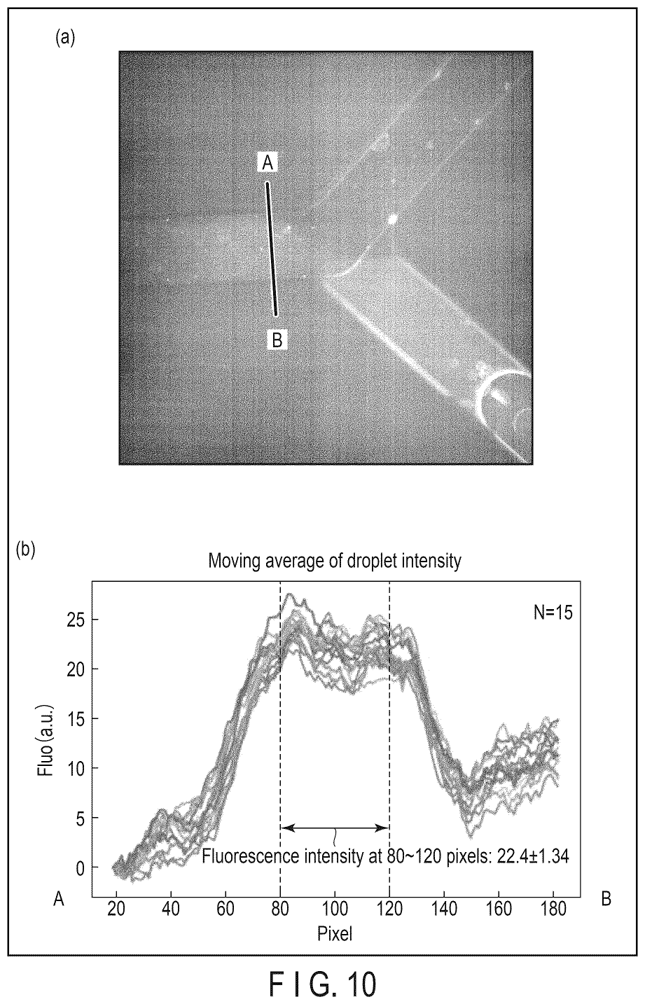

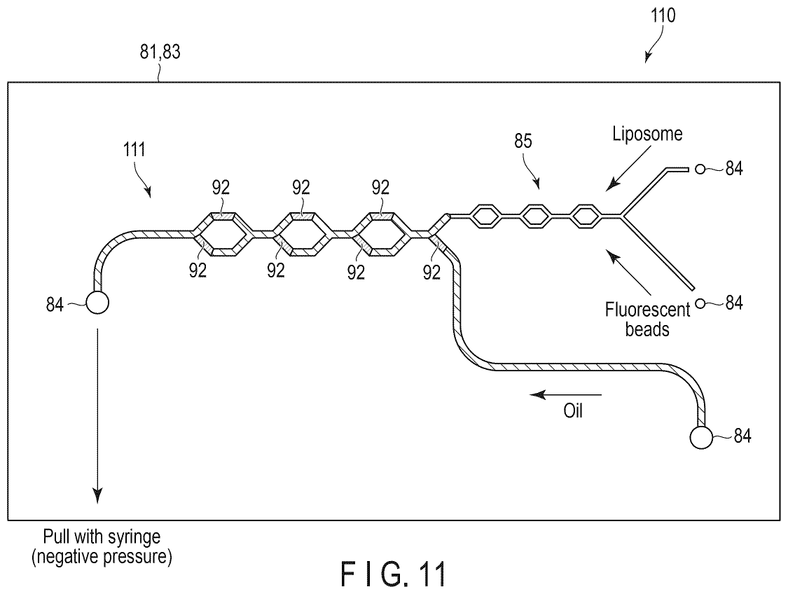

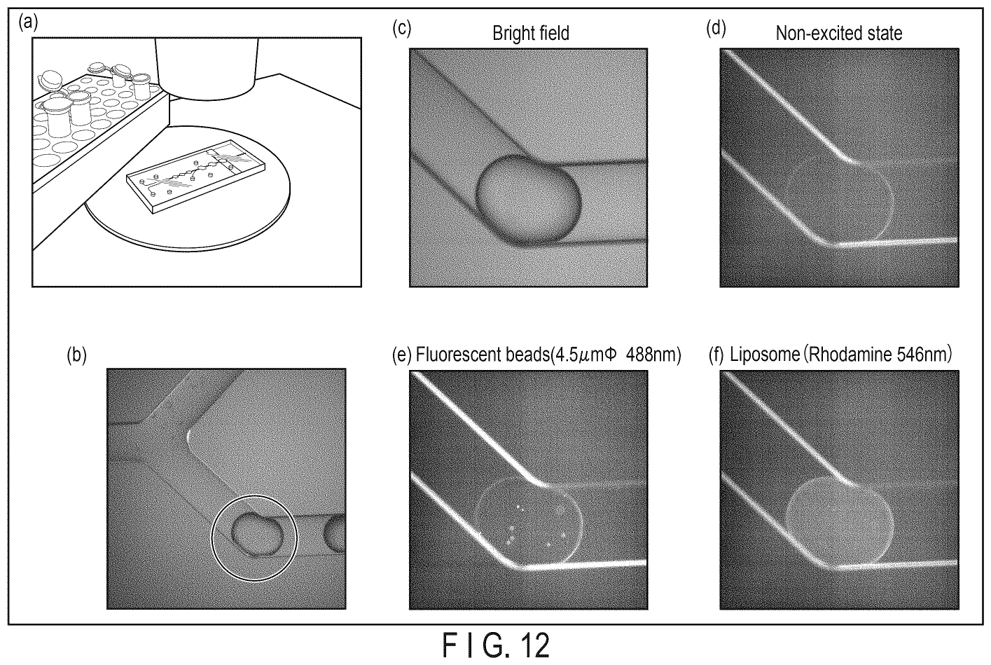

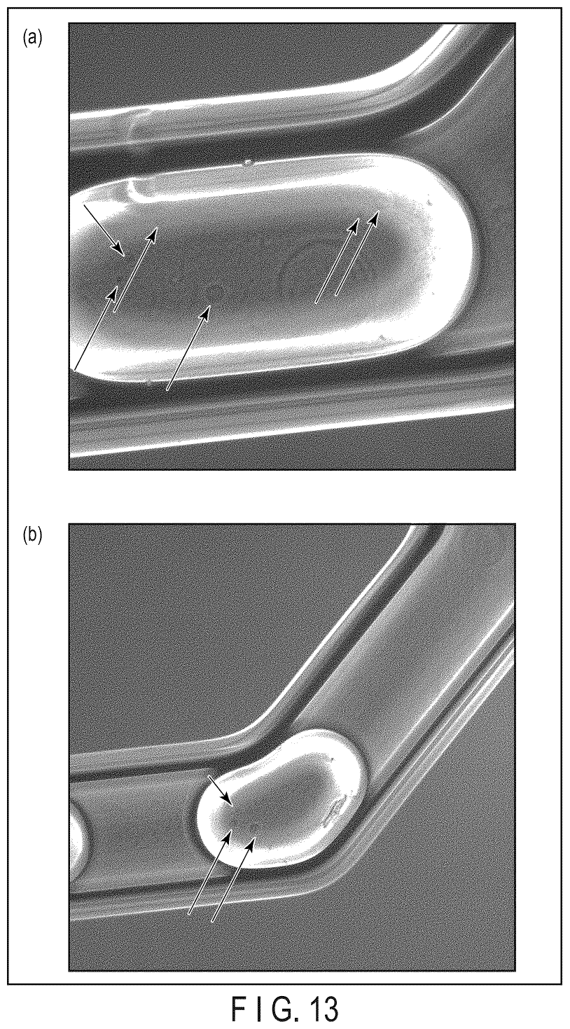

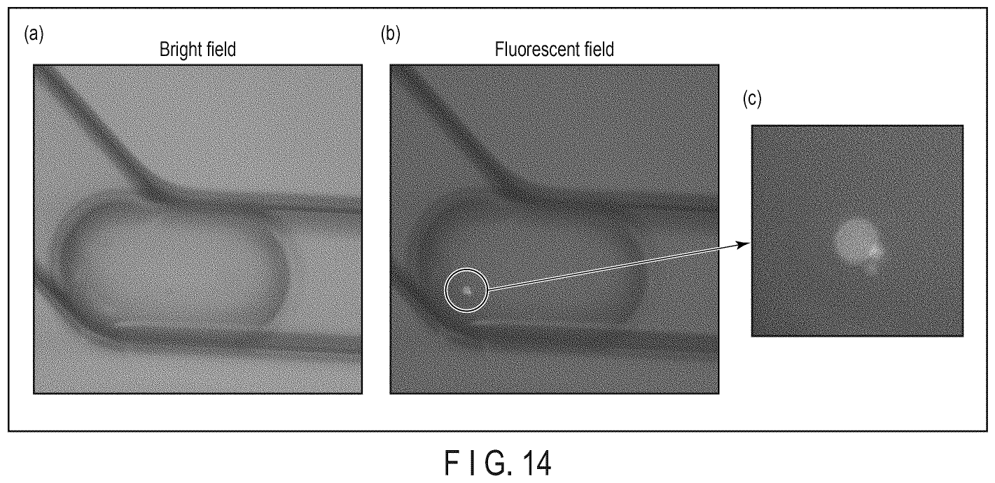

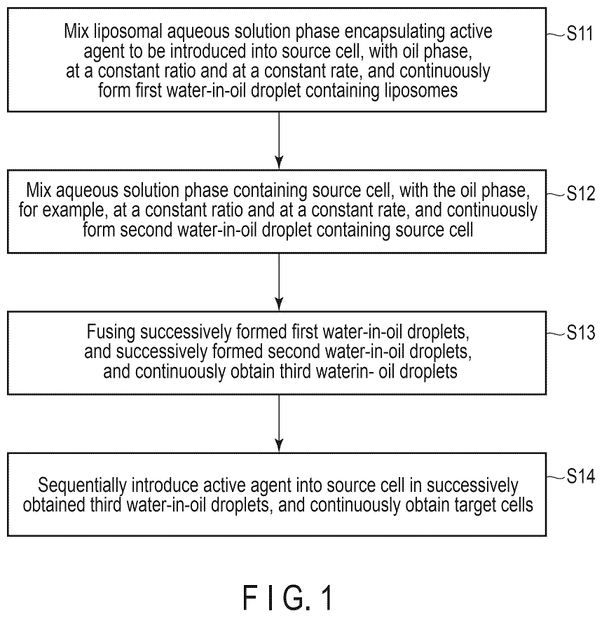

For example, as shown in FIG. 1, this method comprises (a) mixing a liposomal aqueous solution phase encapsulating an active agent to be introduced into source cells, with an oil phase, for example, at a constant ratio and at a constant rate, and continuously forming a first water-in-oil droplet containing liposomes (S11), (b) mixing the aqueous solution phase containing source cells, with the oil phase, for example, at a constant ratio and at a constant rate, and continuously forming a second water-in-oil droplet containing source cells (S12), (c) fusing the successively formed first water-in-oil droplets, and the successively formed second water-in-oil droplets, and continuously obtaining third water-in-oil droplets (S13), and (d) introducing the active agent into the source cells in each of the successively obtained third water-in-oil droplets, and successively obtaining target cells (S14).

A “target cell” is a cell to be produced. An example of a target cell can be any desired cell that, upon introduction of an active agent, is configured to acquire properties different from those of a primary source cell. Such a cell may be, for example, any cell formed by introducing a gene. Examples of target cells can be cells used at the laboratory level, in genetic engineering, in recombinant protein production, in basic research fields, and in medical fields such as gene therapy, cell diagnostics, and regenerative medicine. In the case of cells used in regenerative medicine, examples include induced pluripotent stem cells such as iPS cells and somatic stem cells such as mesenchymal stem cells.

The “source cells” are cells into which an active agent is introduced to form target cells, and are selected according to the target cells to be produced.

The “target cells” can be, for example, cells obtained after contact between the active agent and the source cells, cells obtained after the active agent has acted, cells obtained after reacting with the active agent, or cells obtained after the active agent has been introduced. What target cells are produced and/or recovered may be determined by a practitioner operator as desired. The target cells may be often used as further source cells. In this case, further target cells can be obtained by continued introduction of the active agent. In other words, the target cells are formed from the source cells through a process of causing the source cells to eventually become target cells by applying the active agent into the source cells a plurality of times.

The term “source cells” is used interchangeably with the terms “primary source cells”, “first-stage source cells”, and “seed cells”. For example, when a plurality of active agents are repeatedly introduced into the source cells, properties of the first-stage source cells before introduction of the active agent can change depending on the number of repeated introduction before finally obtaining the target cells. In this case, the term “stage” is used for convenience to describe the state or nature of the source cell or the presence or absence of contact with a specific active agent, and is also described as “source cell in the second stage”, “source cell in the third stage”, . . . “source cell in the n-th stage” (n is an integer of 3 or more), and the like depending on the number of times of introduction of the active agent. Whether the “source cells” brought into contact with the specific active agent are the “source cells in the n-th stage” or the “target cells” may be determined by a practitioner as desired or according to subsequent procedure. Depending on the selected source cells and/or target cells, differentiation is induced by the active agent entering the system containing the source cells a plurality of times to form the desired target cells. In addition, the amount and timing of the entry of the active agent may be controlled according to the selected source cells and/or target cells and as desired.

The “active agent” is any active ingredient that acts on the source cells and/or reacts with the source cells, with actions of changing the source cells into target cells with properties, physical properties and/or morphology different from the primary source cells, introducing the source cells to the target cells, or bringing the source cells into the target cell state. The active agent can be, for example, a substance for recombining the genome of a desired cell, or the like, and can be a substance generally referred to as genes. More specifically, for example, the active agent may be a natural product, compound, extract, nucleic acid, peptide, protein or the like. For example, the nucleic acid can be a nucleic acid fragment, a nucleic acid construct, DNA, RNA or the like. The active agent applied to the source cells may be one type or a combination of two or more types. For uniform introduction, one type of active agent is desirably contained in one liposome. Examples of combinations of a plurality of types of active agents will be further described in the embodiments to be described below.

One typical example of the active agent is a nucleic acid, which may be, for example, a nucleic acid selected from the group consisting of plasmids, oligonucleotides, polynucleotides, small interfering RNA (siRNA), microRNA (miRNA), DNA, aptamers and ribozymes. In addition, the active agent may also be antisense oligonucleotides, antagomir, aDNA, plasmids, ribosomal RNA (rRNA), transfer RNA (tRNA), nuclear small RNA (snRNA), mRNA, and a combination of different types of RNA and/or DNA.

Liposomes can be lipid bilayer membrane particles that encapsulate an aqueous solution core. Liposomes may utilize any liposome known per se. For example, the lipid composition forming the liposome can contain as its components a first lipid (FFT-10) of formula (I) and/or a second lipid (FFT-20) of formula (II). Examples of use of these lipid components will be further described in the embodiments to be described below.

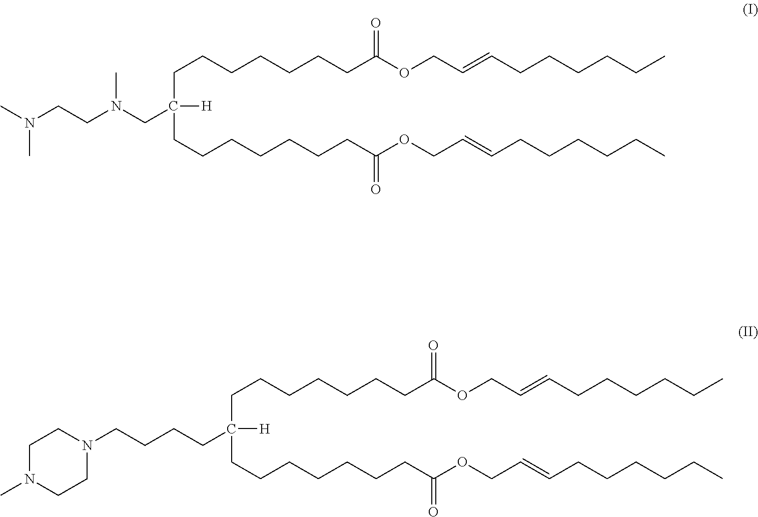

Examples of oils used as the oil phase can be, for example, fluorinated oils, mineral oils with carbon chains, silicone oils, and the like. Examples of fluorinated oils are, for example, fluorinated oils with carbon fluoride, such as Fluorinert. However, fluorinated oils are not limited to these.

Examples of aqueous solutions can be, but are not limited to, buffer solutions, cell culture media, or the like.

Examples of combinations of droplets (aqueous solution phase) and external liquids (oil phase) include, but are not limited to: combinations of aqueous solutions as droplets, and carbon-chained mineral oils, silicone oils, and the like as external liquids; and combinations of aqueous solutions as droplets and fluorinated oils with carbon fluoride, such as Fluorinert, as external liquids.

In such cases, the addition or non-addition of further components such as surfactants, and the like, and their types and amounts of use may be selected in consideration of the combination of oil and aqueous solution. For example, a fluorinated surfactant may also be desirably used in a combination with silicone oil, and the like or a combination with fluorinated oil.

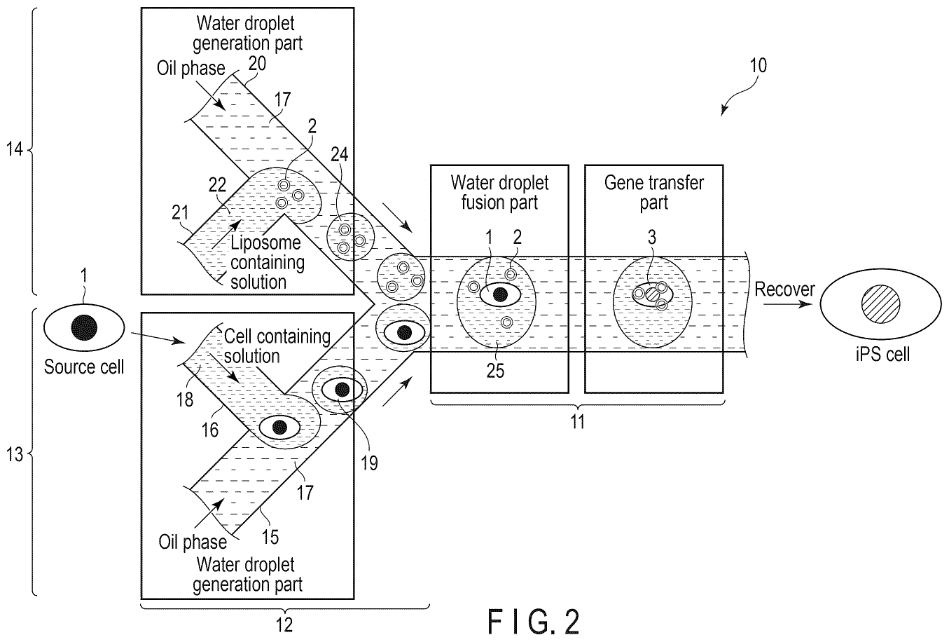

The formation of the first water-in-oil droplets can be executed using, for example, a microfluidic system 10 with three branches as shown in FIG. 2. The microfluidic system 10 comprises microfluidic channels with three branches. In addition, the microfluidic system also includes a main trunk 11 and a branch 12. The branch 12 comprises a first water-in-oil droplet forming channel 13 and a second water-in-oil droplet forming channel 14.

The first water-in-oil droplet forming channel 13 comprises an oil phase feed channel 15 and an aqueous solution phase feed channel 16. The oil phase feed channel 15 and the aqueous solution phase feed channel 16 are connected at right angles, but is not limited to this. Both of the channels may be connected to have the same flow direction or may be connected to have opposite flow directions. However, the channels are desirably connected at right angles in order to generate droplets. The oil phase 17 is fed into the oil phase feed channel 15 through an opening at the end of the channel. The aqueous solution phase feed channel 16 is fed with a cell containing solution 18 from an opening of the channel. The cell containing solution 18 is an aqueous solution containing source cells 1 in suspension. The aqueous solution containing the source cells 1 can be any medium, saline or physiological buffer solution, depending on the cells to be used. For example, by intermittently feeding the cell containing solution 18 into the oil phase 17 at a constant flow rate and/or constant cell concentration, water-in-oil droplets 19 of uniform particle size, which contain source cells uniformly or substantially uniformly, are continuously formed. In other words, by feeding an aqueous solution containing cells at a constant concentration into the oil at a constant flow rate, the water-in-oil droplets 19 have a constant particle size, and the number of cells contained in the water-in-oil droplets 19 is constant.

The second water-in-oil droplet forming channel 14 comprises an oil phase feed channel 20 and an aqueous solution phase feed channel 21. The oil phase feed channel 20 and the aqueous solution phase feed channel 21 are connected at right angles, but is not limited to this. Both of the channels may be connected to have the same flow direction or may be connected to have opposite flow directions. However, the channels are desirably connected at right angles in order to generate droplets. The oil phase 17 is fed into the oil phase feed channel 20 through an opening at the end of the channel. The aqueous solution phase feed channel 21 is fed with a liposome containing solution 22 from an opening of the channel. Liposomes 2 are suspended and contained in the liposome containing solution 22. The aqueous solution containing the liposomes 2 can be any medium, saline or physiological buffer solution, depending on the cells to be used. For example, by intermittently feeding the liposome containing solution 22 into the oil phase 17 at a constant flow rate and/or constant cell concentration, the particle size of the water-in-oil droplets 24 can be constant or substantially constant. In addition, homogeneous water-in-oil droplets 24 that contain the liposomes 2 uniformly or substantially uniformly are thereby continuously formed. In other words, the number of liposomes contained in the water-in-oil droplets 24 is constant by keeping the particle size of the water-in-oil droplets 24 constant or substantially constant. For example, the aqueous solution phase 22 may be fed into the oil phase 17, with the aqueous solution phase 22 and the oil phase 17 merging at the same rate and directed downstream.

The water-in-oil droplets 19 containing the source cells 1 formed intermittently and continuously in the first water-in-oil droplet forming channel 13 and the water-in-oil droplets 24 containing the liposomes 2 formed intermittently and continuously in the second water-in-oil droplet forming channel 14 are fed toward the main trunk 11 from the oil phase 17 in the branch 12, and reach the main trunk 11 at the same or substantially the same timing. Both of the water droplets merge upstream of the main trunk 11, and fuse with each other as a ratio of, for example, 1:1, to form a single droplet. For example, fusion of the first water-in-oil droplets 19 and the second water-in-oil droplets 24 may be caused by changes in conditions in microfluidic channels, for example, merging of the microfluidic channels, changes in pressure in the microfluidic channels, flow in the microfluidic channels, for example, changes in liquid flows, agitation of liquid in the microfluidic channels, and the like. For example, the fusion may be executed between droplets at a constant ratio of, for example, 1:1, and/or at a constant rate, a constant interval, a constant flow rate, and/or a constant volume. The fusion of droplets may be promoted or executed with a droplet fusion mechanism or device that may produce electrical stimulation caused by electrodes, physical stimulation caused by adjusting the width of the channel either a wider or narrower, thermal stimulation caused by a temperature regulator, magnetic stimulation such as a magnetic field caused by a magnetic force, and the like. Alternatively, the water droplet fusion may be controlled by the type and concentration of surfactants to be contained in the aqueous solution of the cell containing solution 18 and/or the liposome containing solution 22.

Subsequently with the water droplet fusion, the active agent (not shown) contained in the liposome 2 is introduced and/or acted upon by the source cell 1 in the water-in-oil droplet. FIG. 2 shows one example in which the active agent contained in the liposome 2 is a gene. The “gene transfer part” in FIG. 2 is one example of an “active agent introduction part”. This term can be reworded according to the type of active agent as appropriate. The introduction of the active agent into the source cell 1 may be promoted by the above-described water droplet fusion mechanism or device. Alternatively, in addition to the water droplet fusion mechanism or device, the introduction may be promoted by an active agent introduction promotion mechanism or device, for example, a gene transfer mechanism or device, an active agent action promotion mechanism or device, or the like, which can cause the electrical stimulation caused by an electrode, the physical stimulation caused by changing the shape of the inner wall surface or inner space of the channel, the thermal stimulation caused by a heater or the like, the magnetic stimulation such as magnetic or magnetic fields caused by a magnetic force, light such as visible light, infrared light and/or ultraviolet light, sound of a specific or arbitrary frequency, sound waves such as ultrasonic waves, and the like. In addition, the introduction of the active agent may also be stimulated or promoted by, for example, temperature control under certain conditions. For example, the introduction of the active agent and/or the active agent action promotion may be executed by adjusting the introduction, action and/or reaction environment of the active agent. Such adjustment of the introduction, action and/or reaction environment of the active agent may be executed by, for example, changing or maintaining at least one environmental condition selected from the time, the temperature and the gas concentration (for example, CO₂ concentration). For example, the active agent may be a substance that acts on the source cell and/or reacts with the source cell in at least one of the time points: before introduction into the source cell, upon contact with the source cell, during the introduction into the source cell, and after the introduction into the source cell. The introduction of an active agent into a cell implies bringing the active agent into a distance where the active agent can act on the cell, bringing the active agent into the cell, bringing the active agent into a state where the active agent can act on the nucleus of the cell, bringing the active agent into the nucleus of the cell, and the like. In addition, the introduction may also indicate that the active agent acts on the cell, that the active agent reacts with the cell, or both. The introduction of the active agent into the cell may be executed by incubation at a constant temperature. The incubation may be executed while being delivered through a flow channel or held in a specific position by, for example, a cell capture mechanism or device. For example, the incubation temperature may be approximately 35° C. to about 39° C., depending on the type of cells and active agents. The active agent is introduced and the source cell 1 becomes a target cell 3. Finally, the target cell 3 is collected.

The direction of movement of the liquid in the microfluidic channel 10 is indicated by an arrow in FIG. 2. The liquid moves from the end on the branch 12 side toward the end on the main trunk 11 side. The movement of the liquid may be caused by a pressure applied from the end on the branch 12 side or by suction from the end on the main trunk 11 side. In other words, such a target cell

preparation method, is, for example, a method of reacting a liposome with a cell in a micro-space, including (1) a process of preparing a water-in-oil droplet containing a cell, (2) a process of preparing a water-in-oil droplet containing a liposome encapsulating an active agent, (3) a process of fusing two water-in-oil droplets, and (4) a process of reacting the active agent encapsulated in the liposome with the cell in the fused water-in-oil droplet. For example, it is possible to prepare a target cell by sequentially introducing a plurality of active agents into source cells. In such a case, for example, (1) through (4) may be repeated as many times as the number of active agents. In such a case, the source cell becomes a target cell as a result of the first activator action or introduction. The obtained target cell may be a second-stage source cell with a different state, properties, and the like from the primary source cell, i.e., at a different stage.

In addition, for example, “sequentially introducing a plurality of active agents into the source cells” may indicate sequentially introducing the same type of active agent into the source cells a plurality of times (two or more times). In this case, (1) through (4) may be repeated as many times as the number of times of introducing the active agent. In addition, repeatedly introducing one type of active agent a plurality of times and sequentially introducing a plurality of types of active agents may be combined as desired.

To summarize the embodiment, for example, using the method, in a microfluidic channel including two branches, each having an end further divided into two branches, oil is poured from one end of one branch to form an oil phase flowing at a constant volume, an aqueous solution containing the source cells is intermittently poured into this oil phase from one end of the other branch in a constant volume and at a constant rate, and a plurality of first water-in-oil droplets containing the source cells can be thereby continuously formed in the oil phase. On the other hand, oil is poured from one end of the other branch to form an oil phase flowing at a constant volume, an aqueous solution containing liposomes is intermittently poured into this oil phase from one end of the other branch in a constant volume and at a constant rate, and a plurality of second oil-in-water-in-oil droplets containing liposomes can be thereby continuously formed in the oil phase. These are merged at good timing, fuse the water droplets are sequentially fused, and the active agent contained in the liposome is introduced. Thus, by encapsulating a certain number of cells and a certain number of liposomes in a uniform microvolume, control of the contact probability and the inhibition of diffusion of liposomes can be implemented simultaneously, thereby improving the rate of introducing the gene to cells. In addition, the cell introduction rate of the active agent becomes uniform, making quality control easier.

In conventional petri dish culture, since it is difficult to uniformly introduce liposomes into each cell, a problem arises that the state of the target cells obtained is varied. However, by reacting a certain number of droplets containing a certain volume of liposomes with, for example, one or more cells, target cells in a uniform state can be produced.

### Second Embodiment

The liposomes used in the first embodiment will be described as a second embodiment. Liposomes are lipid bilayer membrane particles that encapsulate an aqueous solution core, i.e., lipid particles. Liposomes may utilize any liposome known per se. For example, the lipid composition composing the lipid bilayer membrane forming the liposome can contain as its components a first lipid (FFT-10) of formula (I) and/or a second lipid (FFT-20) of formula (II).

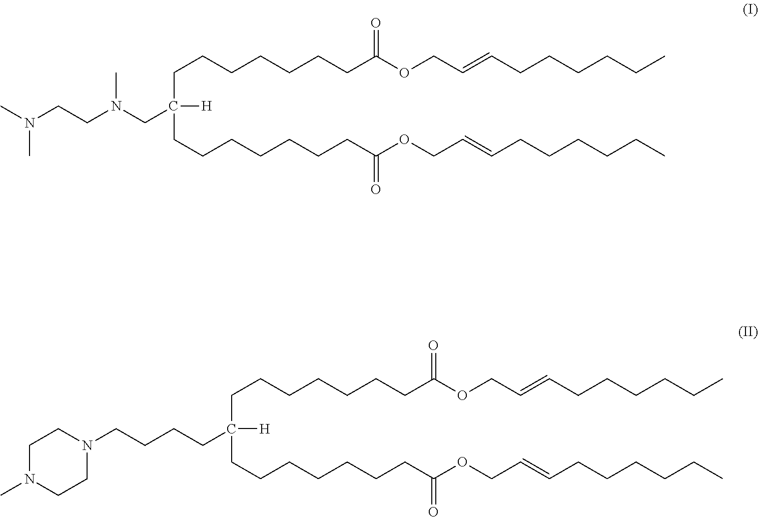

The lipid bilayer membranes, i.e., lipid particles, may further contain lipids in addition to the first lipid and the second lipid described above. A fraction consisting of the first lipid and the second lipid, in the composition of the lipid molecular materials composing the lipid particles, is hereinafter referred to as “first fraction”. In addition, a fraction consisting of the lipid molecular materials other than the first lid and the second lipid is hereinafter referred to as “second fraction”. Lipids in the second fraction are also referred to collectively as “third lipid”.

The terms “first fraction” and “second fraction” refer to the composition of the components of the lipid particles and do not indicate physical locations of the lipids contained therein. For example, the components of each of the first fraction and the second fraction do not need to be one mass in the lipid particles, and the lipids contained in the first fraction and the lipids contained in the second fraction can be mixed to exist. A mixture rate of the first fraction to the total lipid material composing the lipid particles can be, for example, 5% or more, 10% or more, and 15% or more and, for example, 10% to 80%, 15% to 60%, or the like.

In other words, the total content of FFT-10 and FT-20 can be, for example, 5% or more, 8% or more, 10% or more, and 15% or more and, for example, 10% to 80%, 15% to 60%, 15% to 50%, and the like as the mixture rate of the lipid particles. The maximum content of FFT-10 and FFT-20 in the lipid particles can be, for example, the amount by which the lipid particles can form liposomes. The mixture rate of the second lipid in the first fraction can be 0% or more to 100%, for example, 15% to 75%, 20% to 60%, 24% to 50%, and the like. Similarly, the mixture rate of the first lipid in the first fraction may be 0% or more to 100%, for example, 15% to 75%, 20% to 60%, 24% to 50%, and the like.

The particle size of the lipid particles and the penetration to the cells may change depending on the mixture rate of the first lipid to the second lipid in the first fraction. For example, the more the second lipid, the larger the particle size of the lipid particles can be. The average particle size of the lipid particles can be changed depending on the application. For example, the particle size may be adjusted in a range of approximately 50 nm to approximately 300 nm. For example, the particle size may be in a range of approximately 70 nm to approximately 100 nm.

The type of the third lipid in the second fraction of the lipid particles is not limited but, for example, the second fraction contains a base lipid. For example, a lipid that is a major component of biological membranes can be used as the base lipid. The base lipid is phospholipid or sphingolipid, for example, diacylphosphatidylcholine, diacylphosphatidylethanolamine, ceramide, sphingomyelin, dihydrosphingomyelin, kephalin or cerebroside, or a combination thereof.

For example, as the base lipid, 1,2-dioleoyl-sn-glycero-3-phosphoethanolamine (DOPE), 1,2-stearoyl-sn-glycero-3-phosphoethanolamine (DSPE), 1,2-dipalmitoyl-sn-glycero-3-phosphatidylcholine (DPPC), 1-palmitoyl-2-oleoyl-sn-glycero-3-phosphatidylcholine (POPC), 1,2-di-O-octadecyl-3-trimethylammonium propane (DOTMA), 1,2-dioleoyl-3-dimethylammonium propane (DODAP), 1,2-dimyristoyl-3-dimethylammonium propane (14:0 DAP), 1,2-dipalmitoyl-3-dimethylammonium propane (16:0 DAP), 1,2-distearoyl-3-dimethylammonium propane (18:0 DAP), N-(4-carboxybenzyl)-N, N-dimethyl-2, 3-bis (oleoyloxy) propane (DOBAQ), 1,2-dioleoyl-3-trimethylammonium propane (DOTAP), 1,2-dioleoyl-sn-glycero-3-phosphochlorin (DOPC), 1,2-dilinoleoyl-sn-glycero-3-phosphochlorin (DLPC), 1,2-dioleoyl-sn-glycero-3-Phospho-L-serine (DOPS), or cholesterol, or any combination of these, or the like is desirably used.

As the above-described base lipids, cationic or neutral lipids are desirably used particularly, and an acid dissociation constant of the lipid particles can be adjusted depending on their content. DOTAP is desirably used as the cationic lipid, and DOPE is desirably used as the neutral lipid.

The second fraction also desirably contains lipids that prevent aggregation of the lipid particles. For example, the lipids that prevent aggregation may further contain PEG-modified lipids, for example, polyethylene glycol (PEG) dimyristoylglycerol (DMG-PEG), polyamide oligomers (U.S. Pat. No. 6,320,017 B) derived from omega-amino (oligoethylene glycol) alkanoic acid monomer or monosialogangliosides, and the like.

The second fraction may further contain lipids such as relatively less toxic lipids to adjust toxicity; lipids with functional groups that bind ligands to lipid particles; lipids to inhibit leakage of inclusions such as sterol, for example, cholesterol, and the like. In particular, the second fraction desirably contains cholesterol.

The type and composition of the lipids used in the second fraction may be appropriately selected in consideration of the acid dissociation constant (pKa) of the target lipid particles or the particle size of the lipid particles, the type of active agent to be contained, or stability in the cells, and the like.

For example, when containing DOPE, DOTAP, cholesterol, and DMG-PEG, the second fraction is desirable since the delivery efficiency of the active agent is particularly excellent.

In addition to the active agent, further components may be encapsulated in the lipid particles as needed. The further components are, for example, pH adjusters, osmotic pressure adjusters, gene activators, and the like. The pH adjusters are, for example, organic acids such as citric acid, their salts, and the like. The osmotic pressure adjusters are, for example, sugars, amino acids, or the like. In this case, the gene activator can be any substance that promotes or supports the activity of the active agent if the active agent is a gene.

The lipid particles encapsulating the active agent and containing other substances as needed can be produced using publicly known methods used when encapsulating small molecules into the lipid particles, such as the Bangham method, organic solvent extraction method, surfactant removal method, freeze-thaw method, or the like. For example, a lipid mixture obtained by containing the lipid particle material in an organic solvent such as alcohol in a desired ratio and an aqueous buffer solution containing the component to be encapsulated, such as an active agent, are prepared, and the aqueous buffer solution is added to the lipid mixture. The resulting mixture is stirred and suspended, and the lipid particles encapsulating the active agent and the like is thereby formed. The lipid particle thus obtained is one example of the liposomes.

The rate of introducing the gene to cells can be improved by using such liposomes. In addition, the cell introduction rate of the active agent becomes more uniform and the quality can be controlled more easily.

### Third Embodiment

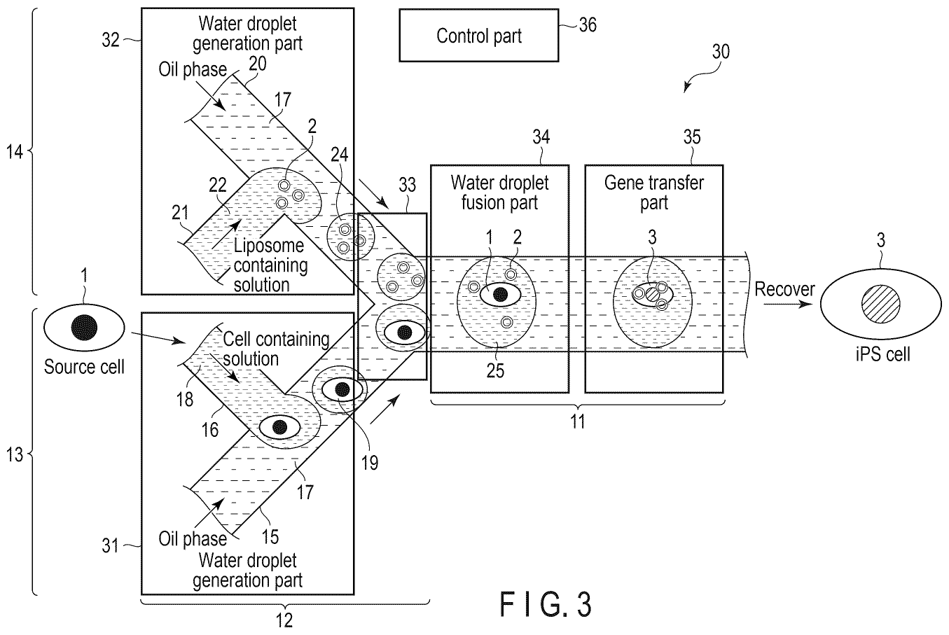

The third embodiment is another example of the microfluidic system described in the first embodiment. One example of the microfluidic system will be described with reference to FIG. 3. A microfluidic system 30 comprises a first water droplet generation part 31, a second water droplet generation part 32, a merging part 33, a water droplet fusion part 34, and an active agent introduction part (gene transfer part) 35. In the first water droplet generating part 31, water-in-oil droplets 19 containing a source cell 1 are formed. In the second water droplet generating part 32, water-in-oil droplets 24 containing liposomes 2 are formed. The merging part 33 is connected to the first water droplet generating part 31 and the second water droplet generating part 32, merging the oil phase containing water droplets 19 containing the source cell 1 and the oil phase containing the water droplets 24 containing liposomes 2. In the water droplet fusion part 34 connected to the merging part 33, the water droplets 19 containing the source cell 1 and the water droplets 24 containing the liposomes 2 are fused in the oil phase. The active agent introduction part (gene transfer part) 35 is connected to the water droplet fusion part 34, receives the fused water droplets from the water droplet fusion part 34 and, for example, incubates them. Thus, the active agent (for example, gene) is introduced into the cell 1 to create the target cell 3.

The microfluidic system 30 creates the target cell by introducing the active agent into the source cell. The microfluidic system 30 comprises microfluidic channels. The first water droplet generation part 31 comprises a first water-in-oil droplet forming channel 13, and also comprises a first oil phase feed channel 15 and a first aqueous solution phase supply channel 16 merging therewith. Oil 17 which becomes an oil phase is fed into the first oil phase feed channel 15 through an opening at the end of the channel. The first aqueous solution phase feed channel 16 is fed with an aqueous solution 18 which becomes an aqueous solution phase from an opening at the end of the channel. The aqueous solution 18 is a source cell suspension and contains source cells.

The second water droplet generation part 32 comprises a second water-in-oil droplet forming channel 14, and also comprises a second oil phase feed channel and a second aqueous solution phase supply channel 21 merging therewith. An aqueous solution 22 supplied to the second aqueous solution phase supply channel 21 from an opening at the end of the channel is a liposome containing solution and contains liposomes encapsulating an active agent.

The merging part 33 is configured such that the first water-in-oil droplet forming channel and the second water-in-oil droplet forming channel 14 merge and are connected to feed water-in-oil droplets 19 from the first water-in-oil droplet forming channel and water-in-oil droplets 24 from the second water-in-oil droplet forming channel to the water droplet fusion part 34. The water droplet fusion part 34 fuses the first water-in-oil droplets 19 and the second water-in-oil droplets 24. A fused water droplet 25 produced by the fusion contains both the liposome 2 and the source cell 1.

The water droplet fusion part 34 comprises a channel leading to an active agent introduction part 35. The active agent introduction part 35 is shown as an example of a gene transfer part 35 in FIG. 3. The active agent introduction part 35 brings the liposome 2 encapsulating the active agent into contact with the source cell 1 to introduce the active agent into the source cell 1. The active agent introduction part 35 may comprise a temperature control mechanism, or incorporate a temperature adjustment mechanism or device arranged to heat or cool the channel of the microfluidic system entirely or partially as desired. The active agent induction part 35 may comprise a condition adjustment mechanism or device which maintains certain conditions until the cells contained therein are transformed into the desired target cells 3. The resulting target cells 3 may be collected through an opening at the end of the channel. Such a microfluidic system can be configured as a channel formed on a substrate of glass, resin, silicon or the like. Alternatively, the microfluidic system may be constituted by a hard or soft tube, or the like.

Furthermore, the microfluidic system 30 may further comprise a mechanism or device for promoting the recovery of the target cells. Such a recovery promotion mechanism or device may be a cell capture mechanism or device, or a mechanism or device that promotes removal of oil from the oil phase and removal of water from the water droplets. For example, physical capture means such as filters, steps, obstructive structures, slits, membranes, and the like may be arranged around the opening.

The water droplet fusion part 34 may comprise a water droplet fusion mechanism or device (not shown) that promotes the fusion of water droplets. Examples of the water droplet fusion mechanism or device may be mechanisms or devices that can produce electrical stimulation caused by electrodes, physical stimulation caused by changing the shape of the inner wall surface or inner cavity of the channel, thermal stimulation caused by heaters or the like, magnetic stimulation of a magnetic field or the like caused by magnetism or a magnetic force, and the like. The water droplet fusion mechanism or device (not shown) may comprise an introduction mechanism or device (not shown) that stimulates or promotes the introduction of the active agent. The water droplet fusion mechanism or device, and the introduction mechanism or device may share one mechanism or device. In addition, the temperature of several parts or the entire body of the microfluidic system may be adjusted by a temperature management mechanism or device, such as a heater and a temperature regulator, as desired.

By flowing the liquid in the channel at a constant rate or applying a constant pressure, using the microfluidic system, the flow rate and/or velocity can also be controlled easily. However, the microfluidic system 30 may further comprise a control part 35 that controls the movement and operation of each part of the microfluidic system. For example, the control part 36 may comprise a movement mechanism (not shown) that is associated with the movement of oil or an aqueous solution in the channel, and a timing adjustment mechanism (not shown) that adjusts the movements in operations and processes executed at each part of the microfluidic system 30. In addition, the control part 36 may comprise a sensor (not shown) for observing or detecting the operations and processes in the microfluidic system 30.

### Fourth Embodiment

The fourth embodiment is an example of a target cell preparation method for sequentially introducing two types of active components. When two types of active components are sequentially introduced into one source cell, for example, the method of the first embodiment may be repeated two times. In this case, the source cell into which the first type of active component is introduced is referred to as a second-stage source cell as a cell different from the primary source cell. In contrast, the primary source cell is also referred to as a first-stage source cell. For example, when the microfluidic system 30 according to the third embodiment is used, it is possible to collect the cells obtained as target cells 3 by the first introduction, further introduce the second active component by use the cells as the second-stage source cells, and finally obtain the desired target cells 3.

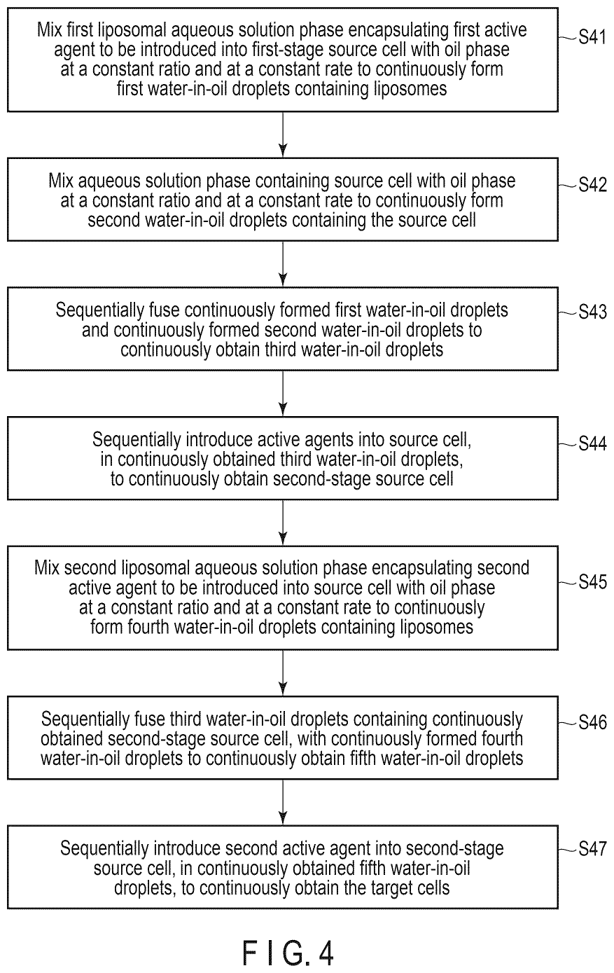

As shown in FIG. 4, the target cell preparation method, which is one example of the fourth embodiment, includes sequentially introducing the first and second active agents into the source cells. For example, the target cell preparation method may comprise the following: (a′) mixing a first liposomal aqueous solution phase encapsulating the first active agent to be introduced into the first-stage source cell with an oil phase at a constant ratio and at a constant rate to continuously form first water-in-oil droplets containing liposomes (S41); (b′) mixing the aqueous solution phase containing a source cell with the oil phase at a constant ratio and at a constant rate to continuously form second water-in-oil droplets containing the source cells (S42); (c′) sequentially fusing the continuously formed first water-in-oil droplets and the continuously formed second water-in-oil droplets to continuously obtain third water-in-oil droplets (S43); (d′) sequentially introducing active agents into the source cells, in the continuously obtained third water-in-oil droplets, to continuously obtain second-stage source cells (S44); (e) mixing a second liposomal aqueous solution phase encapsulating the second active agent to be introduced into the source cells with the oil phase at a constant ratio and at a constant rate to continuously form fourth water-in-oil droplets containing liposomes (S45); (f) sequentially fusing the third water-in-oil droplets containing the continuously obtained second-stage source cells, with the continuously formed fourth water-in-oil droplets to continuously obtain fifth water-in-oil droplets (S46); and (g) sequentially introducing the second active agent into the second-stage source cells, in the continuously obtained fifth water-in-oil droplets, to continuously obtain the target cells (S47).

Such a method is a method of continuously preparing a plurality of target cells, and a plurality of cells into which the active agents are uniformly introduced, can be thereby prepared. In addition, the target cells can be prepared efficiently and/or homogeneously by the system.

### Fifth Embodiment

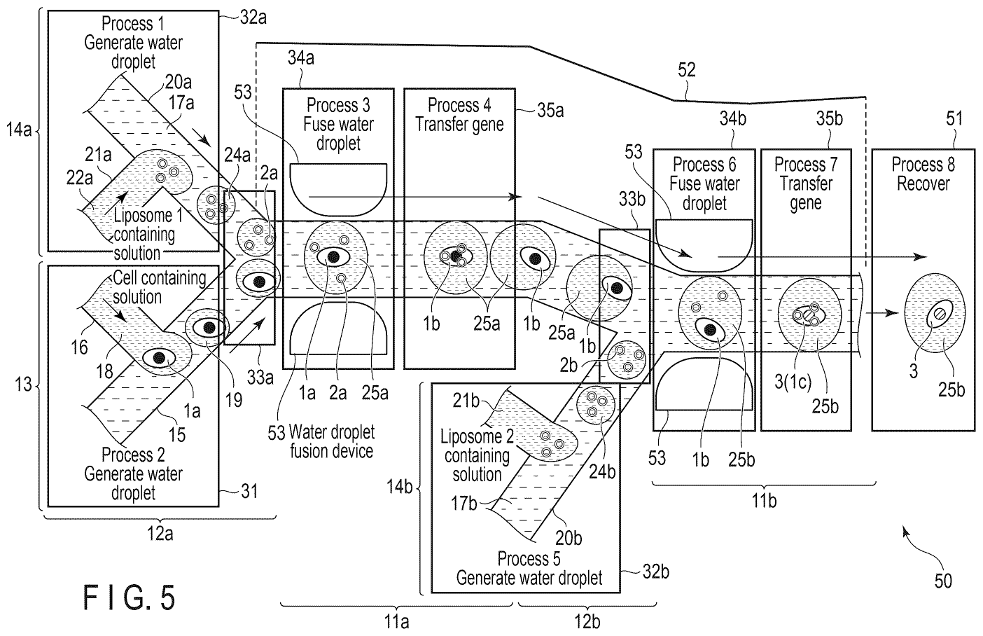

One example of a microfluidic system 50 of the fourth embodiment will be described with reference to FIG. 5. The microfluidic system 50 is a system for preparing target cells by introducing two types of active agents to source cells sequentially, i.e., in two separate stages. Basically, the microfluidic system comprises the configuration of the microfluidic system 30 of the third embodiment, and further comprises a configuration for introducing a second active agent into the cells. Such configurations include a third water droplet generation part 32b, a second-stage water droplet fusion part 34b, an active agent introduction part (gene transfer part) 35b for forming a water-in-oil droplet 24b that contains liposomes 2b containing the second active component. In FIG. 5, a letter “a” is attached to an end of each reference numeral, in the configuration for the first active agent. Since the configuration for the second active agent is basically the same as that for the first active agent, a letter “b” is attached to an end of each reference numeral. In FIG. 5, an example in which the water droplet fusion parts 34a and 34b are arranged to sandwich a channel is shown with any water droplet fusion device used as one example of a stimulation mechanism 53. For example, the stimulation mechanism 53 may be arranged in gene transfer parts 35a and 35b, or in the water droplet fusion parts 34a and 34b and the gene transfer parts 35a and 35b. The microfluidic system can be configured as a channel formed on a substrate of glass, resin, silicon, or the like. Alternatively, the microfluidic system may be constituted by a hard or soft tube, or the like. In addition, in the microfluidic system, a main trunk channel 52 where contact between cells and liposomes and active agent introduction are mainly executed may exist as a backbone supporting the system.

According to the fifth embodiment, it is possible to continuously execute introduction of two types of active agents, i.e., the introduction of first active agents into the first-stage source cells and the introduction of second active agents into the second-stage source cells thereby obtained. Such a system can continuously prepare a plurality of target cells. A plurality of cells into which the active agents are homogeneously introduced can be thereby prepared. In addition, the target cells can be prepared more efficiently and/or with higher quality by the system. For example, by incorporating a control or memory unit such as a computer or CPU, as well as pre-stored programs, tables, timing mechanisms or devices, and timing observation mechanisms or devices, the target cells can be prepared at high throughput, for example, in a fully automated manner.

By conducting the reaction process between the liposomes encapsulating genes and the cells in the water-in-oil droplets, it is possible to inhibit the diffusion, improve the reaction efficiency, and prepare the homogeneous cells. For example, a plurality of genes can be uniformly introduced, such that the reaction of making contact between the liposomes and the cells is made uniform and that the separation of the process of making two different liposomes contact is increased, and, for example, iPS cells can also be prepared homogeneously.

### Sixth Embodiment

The sixth embodiment is an example of a microfluidic system for preparing a target cell by sequentially introducing n types of active agents (where n is an integer of 3 or more) into a source cell in n steps. The target cell can be finally obtained while forming a source cell of the n−1-st stage as intermediates for each introduction of each active agent. Such a microfluidic system may further comprise the number of water droplet generation parts necessary for n types to form water-in-oil droplets for all of n types of active agents used in the microfluidic system 50 shown in FIG. 5, respectively. A basic structure of the microfluidic system 50 is, for example, a channel formed on a substrate. As a whole, one oil-phase channel is a main trunk channel 52 serving as a backbone, and its most upstream side is branched into two channels. One of the branches extends to a water-in-oil droplet forming channel 13 for source cell 1, and the other of the branches extends to a water-in-oil droplet forming channel 14a for a first active agent.

Then, n−1 oil-phase channels 20b to 20n−1 (only 20b shown in the drawing) merge into each other on a downstream side of the main trunk channel 52. On the oil-phase channels, aqueous solution phase channels 21b to 21n (only 21b shown in the drawing) merge into their respective upstream sides. In other words, a water-in-oil droplet forming part 14b for the second active agent to a water-in-oil droplet forming part 14n (not shown) for the n-th active agent merge into the main trunk channel 52 via their respective oil-phase channels. The water-in-oil droplets formed in the respective water-in-oil droplet forming parts are fed to the main trunk channel 52. The source cells 1 contained in the first water-in-oil droplets 19 formed in the uppermost stream are flown in the oil phase in the main trunk channel 52, fed downstream, and sequentially stimulated by the first to n-th active agents. As a result, the stage of the source cell changes sequentially from the initial stage (first stage) to the second and third stages. For example, if the active agent is a gene, n types of genes are sequentially introduced into the source cell and, in some cases, the source cell is transformed. The active agents are introduced into the source cell 1 in several steps, and the target cell 3 is finally obtained.

One example of such a microfluidic system can be configured as follows. The microfluidic system is configured to introduce the first to n-th active agents into the source cell (where n is an integer greater than or equal to 3) and prepare the target cell. The microfluidic system includes a first water-in-oil droplet forming channel, second water-in-oil droplet forming channel, a first fusion part, a first active agent introduction part, third to n+1-th water-in-oil droplet forming channels, second to n-th fusion parts, and second to n-th active agent introduction parts. Such a microfluidic system may repeatedly comprise a minimum repetitive unit “(cell supply 1)-water-in-oil droplet forming channel for liposomes containing active agent-fusion part-active agent introduction part-(cell supply 2)” the desired number of times. In each of the fusion parts, water-in-oil droplets 24a to 24n containing liposomes 2a to 2n, respectively, and water-in-oil droplets 24a to 24n containing source cells 1a to 1n, respectively, are fused to form fused water droplets 25a to 25n. In the fused water droplets 25a to 25n, active agents a to n are introduced into the source cells 1a to 1n to form source cells 1b to 1n of the next stage or form the target cell 3.

Such a microfluidic system comprises, for example, the following configuration: the first water-in-oil droplet forming channel prepares a first water-in-oil droplet containing a cell suspension containing a first-stage source cell, in an aqueous solution phase. The first water-in-oil droplet forming channel comprises a first oil phase feed channel and a first aqueous solution phase feed channel merging thereto; in the second water-in-oil droplet forming channel, a second water-in-oil droplet containing a first liposome encapsulating the first active agent, in the aqueous solution phase, is formed. The second water-in-oil droplet forming channel comprises a second oil phase feed channel and a second aqueous solution phase feed channel merging thereto. The second oil phase feed channel merges with the first oil phase feed channel on the downstream side. The oil phase channels further extend downstream as a backbone structure or main trunk channel from a merging point; the first water-in-oil droplet and the second water-in-oil droplet fuse with each other to form a first fused water droplet, at a first fusion part. The first fusion part is located in an area extending downstream from the merging point of the first water-in-oil droplet forming channel and the second water-in-oil droplet forming channel forming channel. For example, one example of the first fusion part is located downstream from the merging part; at the first active agent introduction part, the first liposome comes into contact with the source cell and the active agent encapsulated in the first liposome is introduced into the source cell. A second-stage source cell is thereby generated. A first active agent introduction part is located downstream of the first fusion part; a third oil phase feed channel for introducing a second active agent is merged downstream of the first active agent introduction part. After that, downstream of the first fusion section, oil phase feed channels for introducing all active agent up to the n-th active agent, respectively, merge the oil phase channel serving as the backbone to extend downstream; third to n+1-th water-in-oil droplet forming channels comprise third to n+1-th oil phase feed channels and third to n+1-th aqueous solution phase feed channels merging therewith, respectively. The third to n+1-th water-in-oil droplet forming channels are located to be sequentially connected to downstream sides of first to n−2-th active agent introduction parts, via the third to n+1-th oil phase feed channels, respectively. Downstream sides of the third to n+1-th oil phase feed channels merge with the main trunk channel. The third to n+1-th water-in-oil droplets are formed in the third to n+1-th water-in-oil droplet forming channels, respectively. The third to n+1-th water-in-oil droplets contain second to n-th liposomes, respectively. The second to n-th liposomes encapsulate second to n-th active agents, respectively; second to n-th fusion parts are arranged downstream of the first to n−1-th active agent introduction parts, respectively. In the second to n-th fusion parts, water-in-oil droplets containing source cells of the second to n−1-th stages from the first to n−1-th active agent introduction parts, and the third to n+1-th water-in-oil droplets containing liposomes containing the second to n-th active agents from the third to n+1-th water-in-oil droplet forming channels, are fused, respectively; in the second to n-th active agent introduction parts connected to the second to n-th fusion parts, the second to n-th liposomes, and the source cells of the second to n−1-th stages, which are contained in the respective fused water droplets, are brought into contact with each other, and the active agents are introduced into the source cells of the stages corresponding thereto. The source cells or target cells of the third to n-th stages are thereby formed. The formed source cells are maintained in water-in-oil droplets and fed to the next introduction. The formed target cell is collected. The target cell may be collected, for example, through an opening provided at the most downstream of the main trunk channel or fed to a collection vessel connected to a downstream side of the main trunk channel and then collected. To prepare for a case where the size of the droplets moving inside the system becomes greater due to the fusion of the water-in-oil droplets, the microfluidic system may comprise a collection mechanism or liquid removal mechanism for temporarily collecting the source cells or removing the liquid from the water droplets. Such a mechanism may be, but are not limited to, means that utilize a shape of a filter, a sieve, a net, a fence, an uneven surface, or the like, means that utilize centrifugal or suction forces, means that utilize electrodes, or magnets, and the like.

According to the embodiment, the reaction efficiency can be improved, the active agents can be introduced uniformly into the source cells, and the homogeneous target cells can be prepared.

### Seventh Embodiment

The seventh embodiment is a microfluidic system in which a channel structure is varied between a fusion part and its upstream and downstream sides, as a fusion mechanism. The embodiment may be the same as the microfluidic system of the other embodiments, except for comprising the fusion structure which has different shapes in the fusion part.

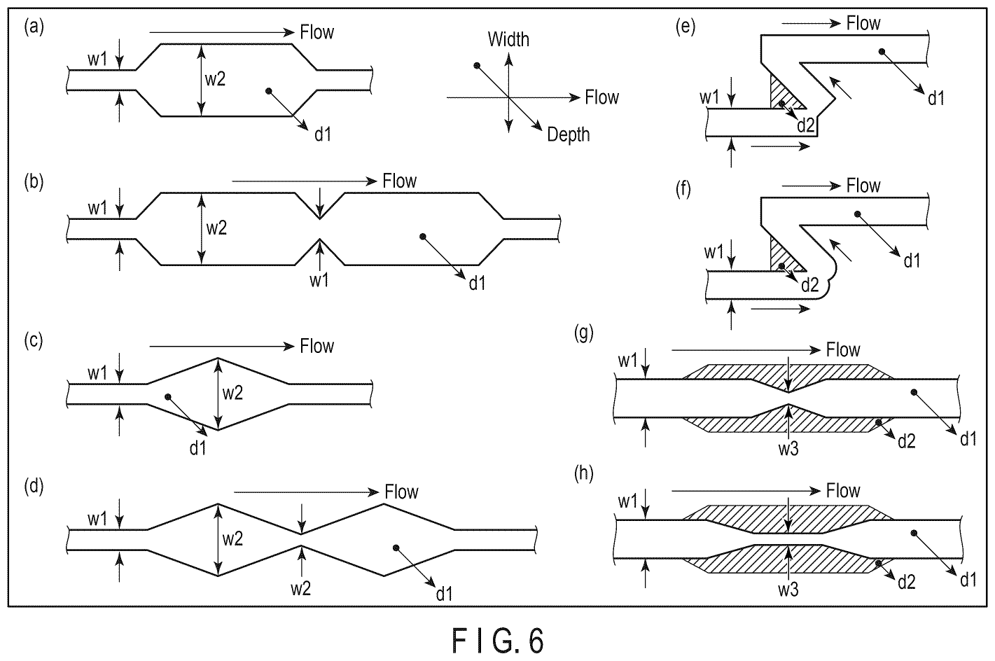

An example of the fusion structure will be described with reference to FIG. 6. FIG. 6 shows a schematic view of the fusion part and the channel portions before and after the fusion part (slightly including its upstream and downstream areas) cut out for convenience and viewed from the top. Part (a) of FIG. 6 shows an example of forming one area having a width (w2) greater than a width (w1) of the channel at the other portion, in the fusion part. In the example of part (a) of FIG. 6, the width w2 is five times longer than the width w1. The inside of the channel extends outward from the ends of the front and rear channels with two trapezoidal bottom sides facing inside. Since the width is temporarily increased, the channel can be made to approach subsequent droplets and the fusion can be promoted. Part (b) of FIG. 6 shows an example in which two extending parts in part (a) of FIG. 6 are arranged longitudinally, i.e., in series in the flow direction of the channel. This shape is also referred to as an hourglass shape. In this case, the channel becomes temporarily wider while approaching subsequent droplets, and then temporarily narrowed, and the pressure can be thereby generated. The fusion is thereby promoted. Part (c) of FIG. 6 shows an example in which extended portions are formed as two triangles (rhombi) having their bottom sides facing each other, in a direction perpendicular to the flow from ends of the front and rear channels, and part (d) of FIG. 6 shows an example in which the rhombi are arranged in series in the flow direction. The same effects as those in part (a) of FIG. 6 and part (b) of FIG. 6 can be obtained from these examples, respectively. In part (e) of FIG. 6 and part (f) of FIG. 6, the channel located in the fusion part is folded twice at 45° C. to form a Z-shape, and the fusion is promoted by applying pressure to water droplets moving inside the channel.

In addition, entry of air bubbles can also be prevented by forming such a shape in the channel. In addition, part (e) of FIG. 6 and part (f) of FIG. 6 also show an example in which a space is formed between channels bending and facing each other at 45 degrees and the space is used as a channel shallower than the other portion. A symmetrical structure in which the flow is aligned can be formed by forming the space.

Alternatively, the channel may be partially made shallow as shown in part (b) of FIG. 6 (part (g) of FIG. 6 and part (h) of FIG. 6). The shallow portion is indicated by hatch lines. A depth d2 of the portion is shallower than a depth d1 of the normal channel. Then, a so-called hourglass structure is formed by reducing a width w3 at a portion of the channel having the normal depth d1. The depth d2 of the area indicated by the hatch lines is effective when, for example, it is approximately one third as long as the depth d1 of the other channels, but is not limited to this. Air bubbles can hardly enter by partially providing the shallow portion. In addition, the fusion can be further promoted by making a portion of the channel narrower and shallower. For example, the fusion can be induced even by providing a step in the depth direction.

### Eighth Embodiment

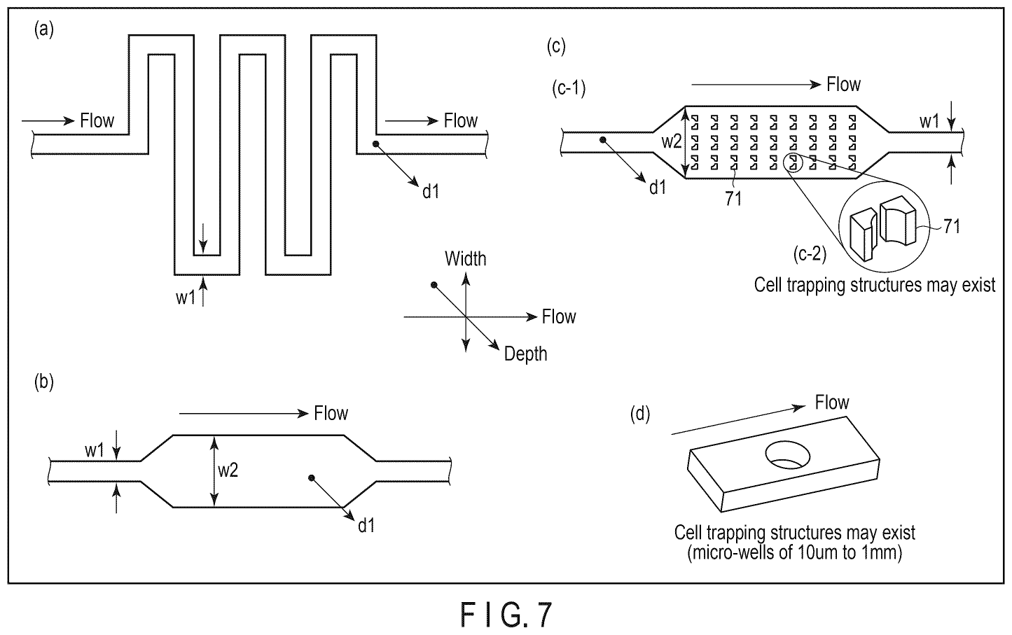

The eighth embodiment is a further example of a flow channel design corresponding to the active agent introduction part as shown in FIG. 7. In an active agent introduction part, an active agent is introduced into source cells by liposomes containing a desired active agent contained in a water-in-oil droplet. For example, in such an active agent introduction part, the introduction of the active agent may be achieved by incubating the fused droplets formed in a fusion part under any conditions. An example of the active agent introduction part comprising such an incubation mechanism is shown in FIG. 7. Constituent elements other than this configuration may be the same as those in any of the above-described embodiments.

The embodiment will be described with reference to FIG. 7. FIG. 7 is a schematic view showing the active agent introduction part and channel portions before and after the part (slightly including its upstream and downstream areas) for convenience as viewed from the upper side. A channel of the active agent introduction part may be shaped in a straight line along the flow direction, as shown in, for example, FIG. 2. However, the incubation time of the cells may be controlled by increasing the channel volume. For example, a meandering structure or a wider structure may be installed. For example, as shown in part (a) of FIG. 7, the channel may be designed to be folded a desired number of times in a direction perpendicular to the flow direction. Alternatively, for example, as shown in part (b) of FIG. 7, a width w2 of a portion of the channel may be greater than a width w1 of the channel in other areas, as shown in part (b) of FIG. 7.

A structure to capture the droplets may be provided as one further example of the incubation mechanism. Examples of a capture structure are shown in part (c) of FIG. 7 and part (d) of FIG. 7. The incubation mechanism shown in part (c) of FIG. 7 is an example in which the channel is partially wider similarly to part (b) of FIG. 7. More specifically, this example is a channel with a trapezoidal-shaped channel space extending from the side surface portion of the channel to both sides in a direction perpendicular to the flow direction, and a plurality of cell trapping structures are arranged on a bottom of the channel (part (c−1) of FIG. 7). The cell trapping structures may be protrusions that facilitate stably trapping cells. In the example in part (c−1) of FIG. 7, two protruding parts having mirror-image contrasting structures are combined with each other to form a single capture unit. In this example, two protruding parts are used as one set, and nine sets in the flow direction and three sets are arranged in the direction perpendicular to the flow. Part (c−2) of FIG. 7 is an enlarged perspective view showing one set of the cell trapping structures shown in part (c−1) of FIG. 7. one protrusion is shaped in a rectangular parallelepiped having a partially cutaway portion and long in the depth direction. When the two protrusions are aligned as a capturing unit, the cutaway portions form a concave portion obtained by dividing a column in an axial direction. Cells are stably trapped at this portion. However, the specific shape is not limited to this. In addition, for example, the cell trapping structure is not arranged only on the bottom surface (or lower surface) of the channel, but may be arranged on the ceiling (upper surface) or both sides of the channel. For example, to prevent droplets from escaping the well, appropriate arrangement may be selected depending on the combination of droplets and solvents. For example, the cell trapping structure is desirably arranged on the lower surface when a solution with a lighter specific gravity than an aqueous solution, such as mineral oil, is used, and the cell trapping structure is desirably arranged on an upper surface when a solution with a heavier specific gravity than the aqueous solution, such as Fluorinert, is used. In such way, the position of the droplets can be fixed. The size of the trapping structure may be varied depending on the size of the droplets to be prepared. For example, the trapping structure may be designed such that the diameter of one capture unit is approximately 10 μm to approximately 1 mm. In addition, for example, the incubation mechanism may be a recess formed in the bottom surface of the channel. Such a recess may be a micro-well structure or a plurality of micro fragments forming micro-wells may be arranged. For example, the size of one micro-well may be approximately 10 μm to approximately 1 mm in diameter. An example of the arrangement of the micro-wells may be, but is not limited to, the arrangement shown in the set of projections in part (c−1) of FIG. 7. The stable and efficient reactions can be made by the above-described embodiment.

### Ninth Embodiment

The ninth embodiment is a method of continuously introducing an active agent into source cells. This method comprises forming a first water-in-oil droplet containing source cells by mixing an aqueous solution containing source cells with an oil phase, forming a second water-in-oil droplet containing liposomes by mixing a liposomal aqueous solution encapsulating an active agent with an oil phase, fusing the formed first water-in-oil droplet with the second water-in-oil droplet, and introducing the active agent into the source cells in the fused droplet. The details have been described above. In addition, for example, a plurality of active agents can be sequentially introduced into one cell by repeating some or all of these processes as described above. Alternatively, the same type of active agent can be introduced a plurality of times. Furthermore, the type of liposome to be used can be changed for each introduction and each active agent. The formation of the water-in-oil droplets can be executed by merging each phase into the microfluidic channel and further using their merging. For example, a swirl channel is desirably used in this case. The swirl indicates, for example, a swirl flow that occurs around a cylinder axis in a piston mechanism, but a microfluidic channel in which such a swirl flow occurs may be merged. According to the embodiment, the reaction efficiency can be improved, the active agents can be introduced uniformly into the source cells, and the homogeneous target cells can be prepared.

### EXPERIMENTAL EXAMPLES

### Example 1 Formation Test of Water-In-Oil Droplets by Microfluidic System

### (1) Preparation of Microfluidic System

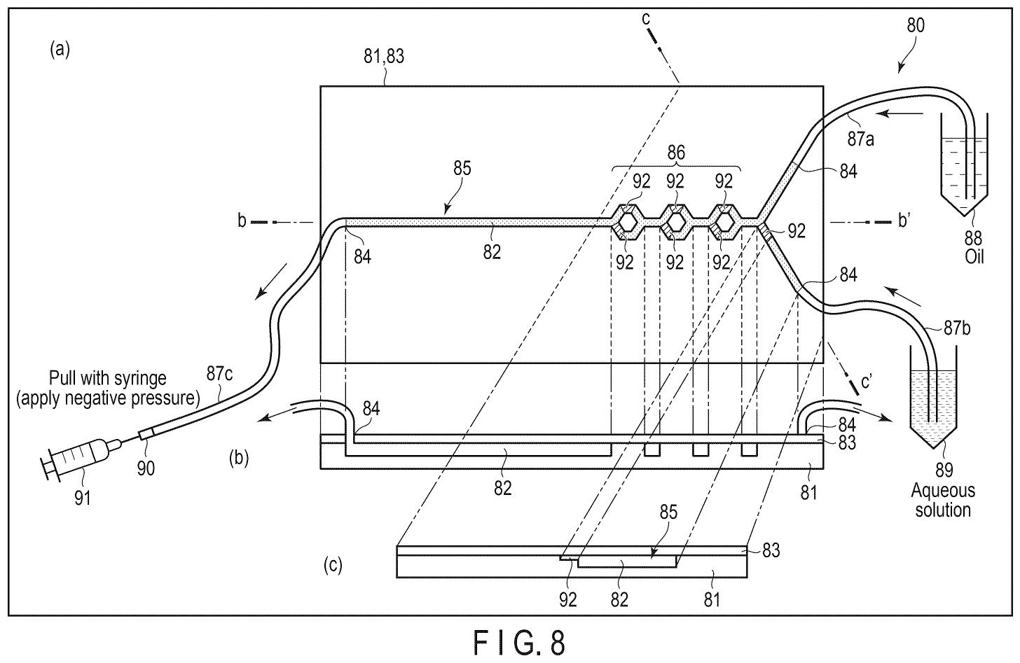

Water-in-oil droplets were formed using the microfluidic system. First, a microfluidic system 80 was constructed as shown in FIG. 8. part (a) of FIG. 8 is a schematic plan view showing the microfluidic system 80, part (b) of FIG. 8 is a cross-sectional view showing the microfluidic system 80 in part (a) of FIG. 8 as cut along line b-b′, and part (c) of FIG. 8 is a cross-sectional view showing the microfluidic system 80 of part (a) of FIG. 8 as cut along line c-c′. The microfluidic system 80 is constituted by overlapping two substrates. One of the substrates is a substrate 81. The substrate 81 is a glass plate having a size of 5 cm×3 cm×3 mm. The substrates 81 has a groove 82 with a depth of 200 μm formed on one side of the substrate 81. The substrate 81 comprises a further substrate of the same size as the substrate 81 on the groove side face, as a lid 83. The substrate 81 and lid 83 are constructed by overlapping such that they are bonded or fixed to each other. Openings 84 facing upward were provided at both end portions of the groove. A channel 85 is defined by the shape of the groove 82. The flow channel 85 comprises an end divided into two portions, an annular structure 86 in which three continuous hexagons are provided downstream from the merging point of the divided portions and arranged in series in the flow direction, a downstream straight structure, and openings 84 at the respective ends of the lid 83. The hexagonal annular structure 86 is a swirl channel. Syringe tubes 87 are attached to edges of three openings 84 of the lid 83, respectively. A tube 87a extending from one of the upstream openings 84 leads to an interior of a container 88 containing the oil, and a tube 87b extending from the other upstream opening 84 leads to an interior of a container 89 containing the aqueous solution. A tube 87c extending from a downstream opening 84 is connected to a cylindrical end of a syringe 81 via a joint 90. By the way, a liquid feed mechanism or device such as a peristaltic pump may be used instead of a syringe to accomplish feeding the liquid in the channel.

As shown in part (c) of FIG. 8, the channel 85 includes seven areas 92 that are partially shallow. Such areas may be made to have a smaller depth and to be shallower than areas of the other grooves when the groove is formed in the substrate. Thus, the area 92 which has a smaller depth, i.e., shallower than the other areas can be obtained. Mixing the liquids is often conducted by providing the shallow area 92. Such an area 92 is provided in the channel on the aqueous solution side just before the aqueous solution side branch and the oil side branch merge. The other six areas 92 are provided in the annular structure 86. The oil from the container 88 and the aqueous solution from the container 89 are mixed to form a water-in-oil droplet. By the way, the annular region 86 can also be used as a water droplet fusion mechanism in the embodiment described above. A shallow area 92 is provided on two non-adjacent sides of the hexagon constituting the annular structure 86.

### (2) Liposome Water-In-Oil Droplet Formation Test

0.1% (molar ratio) Rhodamine PE (produced by Avanti) were added to No. 43 lipid composition to be visualized with fluorescence, suspended in HEPES solution to 5%, and this solution was considered as an aqueous solution of the liposome suspension and used as an aqueous solution phase. FC40 (Fluorinert, produced by 3M) was prepared as the oil phase. These were contained in containers 88 and 89, respectively. A plunger of a syringe 81 was pulled, and each solution was fed while making the pressure in the channel negative, and the inside of the channel 85 was observed from above with an optical microscope for bright field and fluorescent field (wavelength: 546 nm).

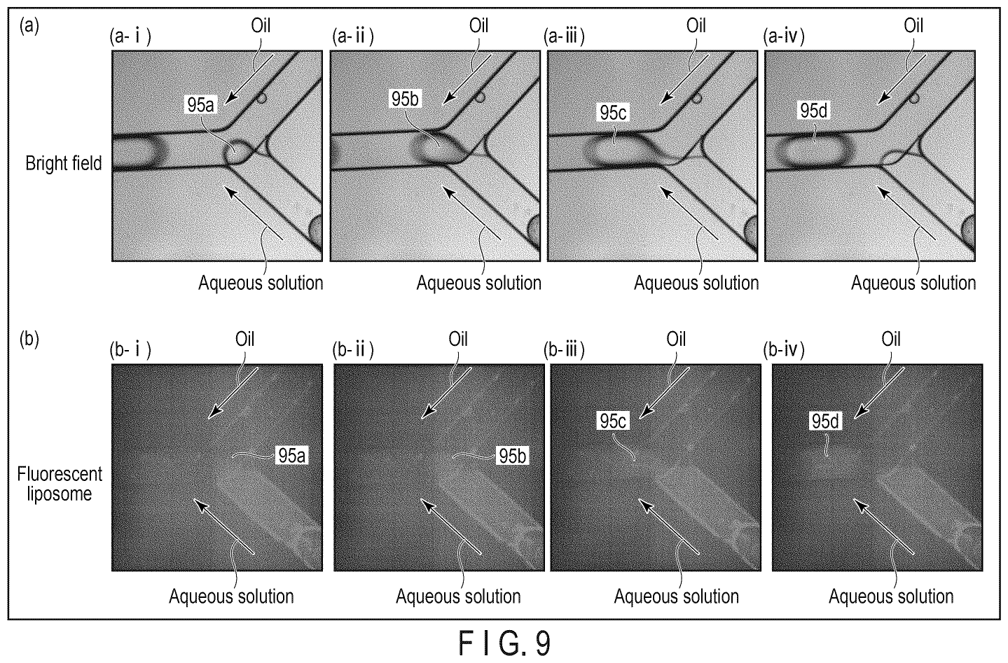

The results are shown in FIG. 9. The observation area was the area located just after the merging immediately before the merging of the oil phase and the aqueous solution phase in the upstream side of the channel. The inner diameter of the channel is 600 μm. part (a) of FIG. 9(a) shows a group of images captured over time in bright field, and part (b) of FIG. 9 shows a group of images captured over time in fluorescent field. Arrows in the drawings indicate the direction of oil flow and the direction of aqueous solution flow, respectively. By pulling the plunger of the syringe 91, statuses of the oil phase from the container 88 and the liposome suspension solution from the container 89 moving downstream in the channel 85 at a constant rate were observed. Along with this, statuses of water droplets generated toward the oil phase as a water-in-oil droplet 95a (part (a-i) and (b-i) of FIG. 9), gradually swelling 95b (part (a-ii) and (b-ii) of FIG. 9), growing 95c (part (a-iii) and (b-iii) of FIG. 9), becoming independent of the matrix aqueous solution, and finally becoming a single droplet (water droplet) 95d in oil (part (a-iv) and (b-iv) of FIG. 9), were clearly observed. It was indicated by this result that water-in-oil droplets can be formed using a microfluidic structure.

### (3) Uniformity Test of Water-In-Oil Droplets

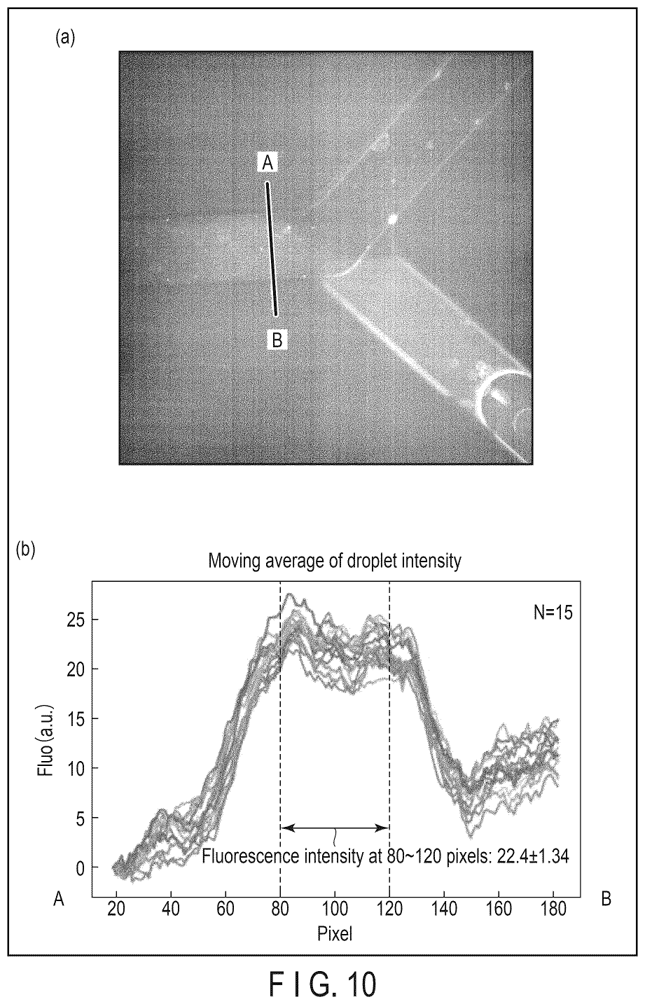

Then, water-in-oil droplets were continuously formed by the same method as (2) described above, and the fluorescence intensity of each of the droplets was observed. The results are shown in FIG. 10. Part (a) of FIG. 10 shows an image captured under a mirror in the fluorescent field. By analyzing this image, a moving average of the fluorescence intensity and plus and minus 20 pixels in the cross-section in line A-B in part (a) of FIG. 10(a) was obtained for the fluorescence intensity of water-in-oil droplets in fifteen cases in the channel. In part (b) of FIG. 10, the obtained fluorescence intensity is plotted on the vertical axis and the pixel position information corresponding to line A-B is plotted on the horizontal axis. In addition, the data were corrected by moving downward the graph parallel to the direction of the vertical axis such that the initial value became zero. As a result, it was clarified that the fluorescence intensity (i.e., liposome amount) of each droplet (i.e., water-in-oil droplet) was substantially equal. The fluorescence intensity at 80 to 120 pixels was 22.4±1.34. It was clarified by these results that the water-in-oil droplets with uniform particle size can be formed by the microfluidic system and that liposomes can be uniformly encapsulated in a plurality of water-in-oil droplets.

Example 2 Simultaneous encapsulation of fluorescent beads and liposomes in water-in-oil droplets in microfluidic system

### (1) Preparation of Microfluidic System and Test of Simultaneous Encapsulation in Water-In-Oil Droplets

A test to confirm that two types of components, i.e., fluorescent beads and liposomes, can be simultaneously encapsulated in water-in-oil droplets was conducted using the microfluidic system. Fluorescent beads were used as cells since they had the same size as a normal cell.

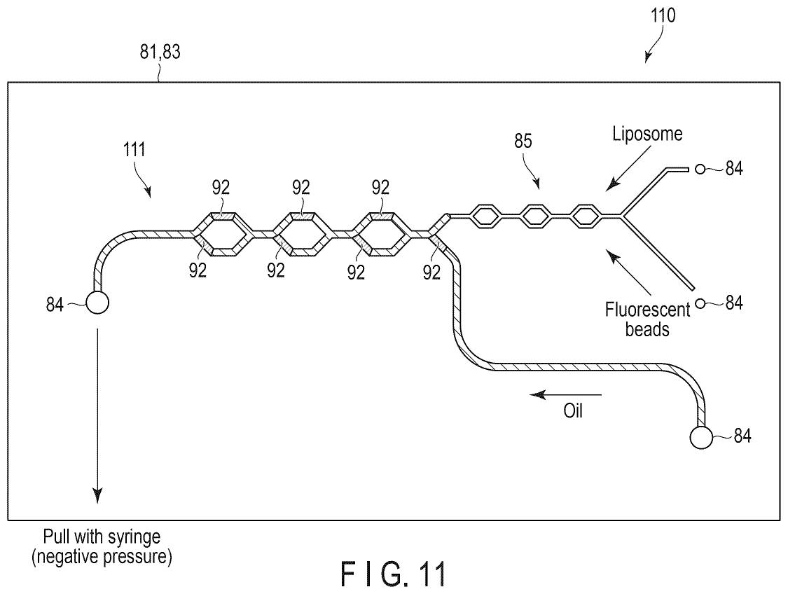

The microfluidic system used is shown in FIG. 11. A microfluidic system 110 extends to the downstream area of the annular structure 86 (one example of a swirl channel) of the channel 85 in the microfluidic system 80 shown in FIG. 8 (part (a) of FIG. 8), and a further oil phase channel is connected to the system. A channel 111 of the microfluidic system 110 comprises a structure provided from the channel 85 to a slightly downstream side of the annular structure 86, and a further annular structure connected to the structure, at the upstream. This further annular structure is also a swirl channel in which seven shallow areas 92 are installed, similarly to the annular structure 86. The flow of liquid into the channel 111 is as follows. First, an aqueous solution phase containing liposomes suspended in an aqueous solution and an aqueous solution phase containing suspended fluorescent beads are mixed through annular structure 86, in the area corresponding to the channel 85, and are fed downstream. The aqueous solution merged with the oil further fed in the oil phase channel and passed through the shallow area 92 and the further annular structure, and water-in-oil droplets were thereby formed.

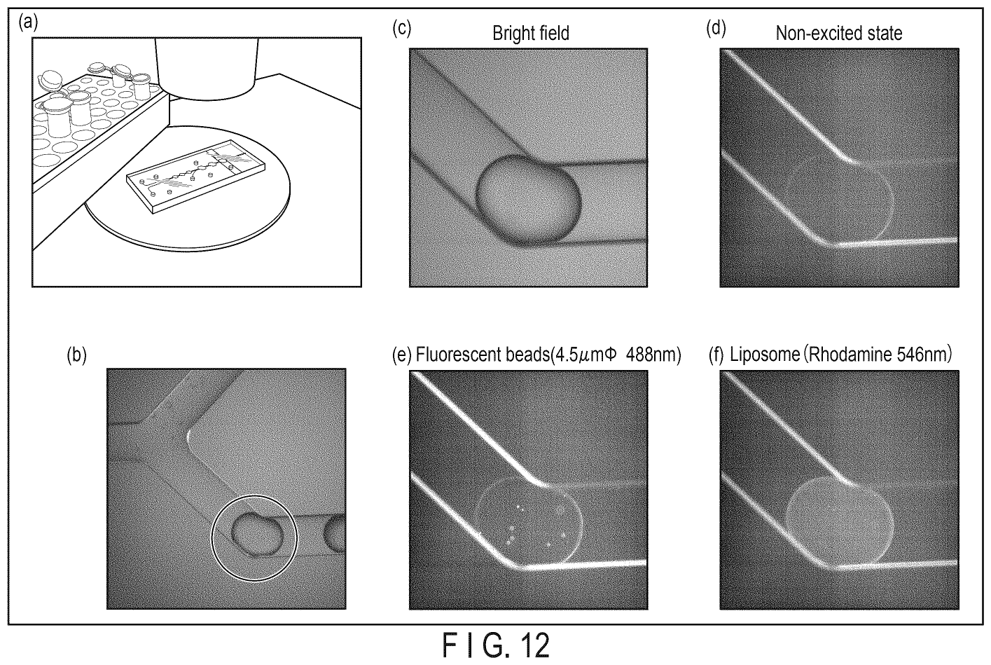

After the above water-in-oil droplets were formed, the portion of the further annular structure (swirl channel) was observed under a microscope in bright field and fluorescent field. The results are shown in FIG. 12. Part (a) of FIG. 12 shows an image of the experiment including the appearance of the microfluidic channel 110 used in the test. Part (b) of FIG. 12 shows an image of the fused water droplet remaining in one corner of the hexagon of the swirl channel. The water-in-oil droplet is circled. The enlarged images of this water-in-oil droplet are shown in part (c) of FIG. 12 to part (f) of FIG. 12. Part (c) of FIG. 12 shows an image of the same site as that in part (b) of FIG. 12 observed in bright field. A well-defined water-in-oil droplet was observed. Part (d) of FIG. 12 shows an image of the same site further observed with fluorescence under non-excited state. The contour of the water-in-oil droplet seems to produce fluorescence. Part (e) of FIG. 12 shows an image of observed fluorescent beads producing green fluorescence in the excited state. As is obvious from this figure, the fluorescent beads contained in the water-in-oil droplet could be observed. Part (f) of FIG. 12 shows an image of an observed Rhodamine liposome producing red fluorescence. It was found that Rhodamine was dispersed throughout the water-in-oil droplet. It could be confirmed by these results that the fluorescent beads (4.5 μmφ) imitating the cells and Rhodamine imitates the liposomes can be simultaneously encapsulated in the water-in-oil droplets by the microfluidic system comprising the microfluidic channel 110.

### Example 3 Test of Encapsulation of Artificial Cells in Water-In-Oil Droplets

The same microfluidic system 80 as that used in the test of Example 1 was prepared. GUV with a lipid composition of 100% DOPC were prepared as artificial cells. Sucrose 100 mM aqueous solution was used as the internal fluid. The artificial cells were suspended in a 100 mM glucose solution. The aqueous solution in which the artificial cells were suspended was fed into channel 85 as the aqueous solution phase, together with Florinert serving as the oil phase. The feeding was conducted by pulling a plunger of a syringe 91 attached to the downstream opening and thereby sucking from the upstream opening into the channel 85.

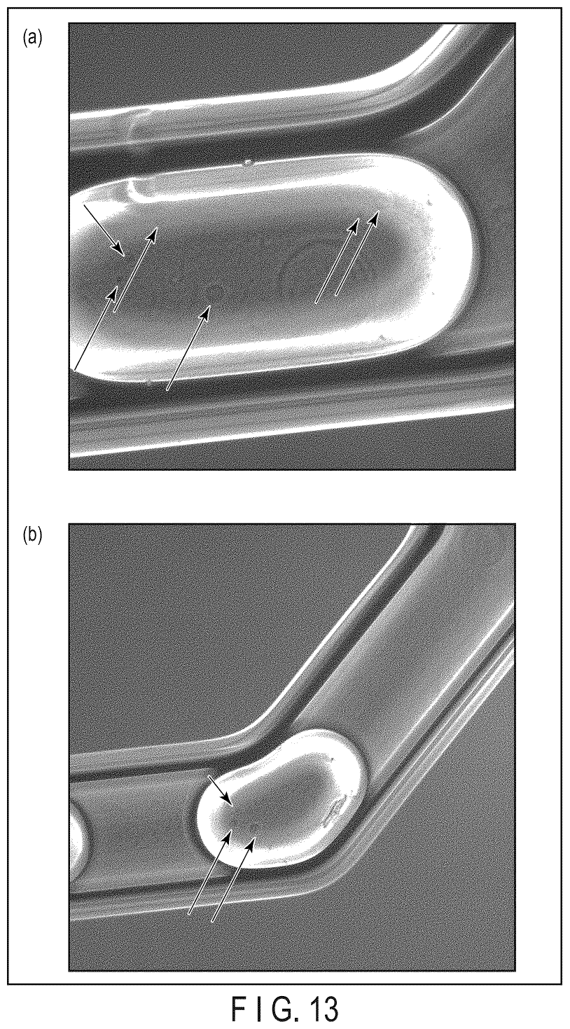

After the channel 85 was filled with the liquid, a corner of the annular structure 86 (swirl channel) was observed under a microscope. The results are shown in FIG. 13. Part (a) of FIG. 13 and part (b) of FIG. 13 show images of the water droplets that formed and existed in different positions. The GUV lipid composition (artificial cells) in the water droplets are indicated by arrows. As a result, it was clarified that artificial cells can be encapsulated in the water-in-oil droplets by the microfluidic system.

### Example 4 Test of Encapsulation of Artificial Cells in Water-In-Oil Droplets

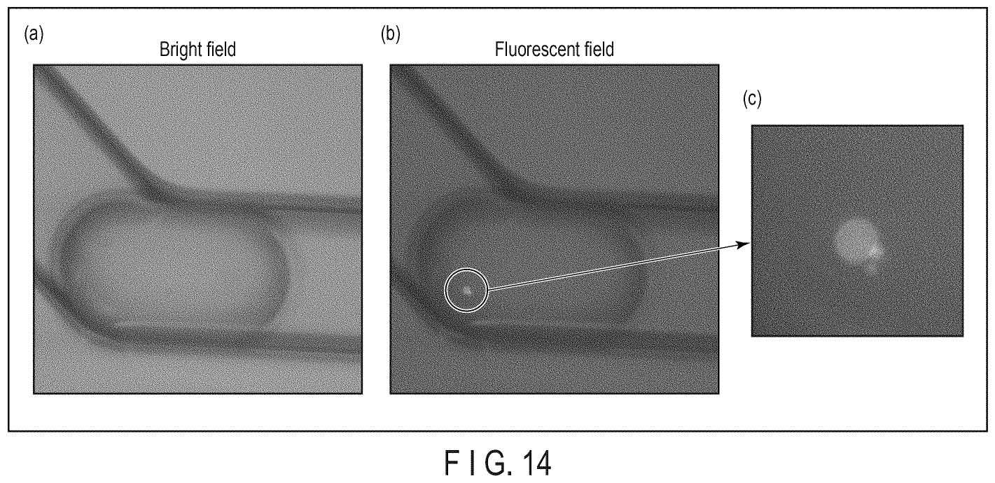

GUV were prepared as artificial cells with a lipid composition of 99.9% DOPC and 0.1% Rhodamine (Rho-PE). Sucrose 200 mM solution was used as the internal fluid. The artificial cells were suspended in a 200 mM glucose solution. The aqueous solution in which the artificial cells were suspended was fed into channel 85 as the aqueous solution phase, together with Florinert serving as the oil phase. The test was conducted in the same manner as Example 3. The results are shown in FIG. 14. When a corner of the annular structure 86 (swirl channel) was observed under a microscope, the water-in-oil droplet was observed in both the non-fluorescent excited state (part (a) of FIG. 14) and the fluorescent excited stated (part (b) of FIG. 14). When fluorescence was observed, fluorescence generated from Rhodamine could be observed. As a result, it was clarified that artificial cells can be encapsulated in the water-in-oil droplets by the microfluidic system.

It was clarified by these results that the cells and/or liposomes can be encapsulated in the water-in-oil droplets by the microfluidic system. It was suggested that the active agent can be uniformly introduced into the cells by using the microfluidic system. In addition, it was also suggested that a plurality of active agents can be thereby uniformly introduced into the cells. Thus, it was suggested that techniques to improve the reaction efficiency, introduce the active agent uniformly into the source cells, and produce homogeneous target cells can be provided.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Examples of further embodiments will be described below.
(1) A target cell preparation method comprising continuously introducing an active agent into a source cell, the method comprising:
forming a first water-in-oil droplet containing the source cell by mixing an aqueous solution containing the source cell with an oil phase;
forming a second water-in-oil droplet containing a liposome by mixing a liposomal aqueous solution encapsulating the active agent with an oil phase;
fusing the formed first water-in-oil droplet with the second water-in-oil droplet; and
introducing the active agent into the source cells in the fused water droplet.
(2) A target cell preparation method comprising introducing an active agent into a source cell, the method comprising:
(a) mixing a liposomal aqueous solution phase encapsulating an active agent to be introduced into the source cell, and an oil phase at a constant ratio and at a constant rate, to continuously form a first water-in-oil droplet containing the liposome;
(b) mixing the aqueous solution phase containing the source cell, and the oil phase, to continuously form a second water-in-oil droplet containing the source cell;
(c) sequentially fusing the successively formed first water-in-oil droplet, and the successively formed second water-in-oil droplet, to continuously obtain third water-in-oil droplets; and
(d) sequentially introducing the active agent into the source cell in each of the continuously obtained third water-in-oil droplets, to continuously obtain the target cell.
(3) A target cell preparation method comprising sequentially introducing a first active agent and a second active agent into a source cell, the method comprising:
(a′) mixing a first liposomal aqueous solution phase encapsulating the first active agent to be introduced into the source cell of a first stage with an oil phase to continuously form a first water-in-oil droplet containing the liposome;
(b′) mixing the aqueous solution phase containing the source cell with the oil phase at a constant ratio and at a constant rate to continuously form a second water-in-oil droplet containing the source cell;
(c′) sequentially fusing the continuously formed first water-in-oil droplet and the continuously formed second water-in-oil droplet to continuously obtain a third water-in-oil droplet;
(d′) sequentially introducing the active agent into the source cell, in the continuously obtained third water-in-oil droplet, to continuously obtain a second-stage source cell;
(e) mixing a second liposomal aqueous solution phase encapsulating the second active agent to be introduced into the source cell with the oil phase at a constant ratio and at a constant rate, to continuously form a fourth water-in-oil droplet containing the liposome;
(f) successively fusing the third water-in-oil droplet containing the continuously obtained second-stage source cell with the continuously formed fourth water-in-oil droplet to continuously obtain fifth water-in-oil droplets; and
(g) sequentially introducing the second active agent into the second-stage source cell, in each of the continuously obtained fifth water-in-oil droplets, to continuously obtain the target cell.
(4) A target cell preparation method comprising sequentially introducing all of first to n-th active agents (where n is an integer larger than or equal to 3) into a source cell, the method comprising:
(a″) mixing a first liposomal aqueous solution phase encapsulating the first active agent to be introduced into the source cell of a first stage with an oil phase to continuously form a first water-in-oil droplet containing the liposome;
(b″) mixing an aqueous solution phase containing the source cell of the first stage with the oil phase at a constant ratio and at a constant rate to continuously form a second water-in-oil droplet containing the source cell;
(c″) sequentially fusing the continuously formed first water-in-oil droplet and the continuously formed second water-in-oil droplet to continuously obtain a third water-in-oil droplet;
(d″) sequentially introducing the active agent into the source cell, in the continuously obtained third water-in-oil droplet, to continuously obtain the source cell of a second stage;
(e′) mixing an n−1-th liposomal aqueous solution phase encapsulating an n−1-th active agent to be introduced into the source cell with the oil phase to continuously form an n+1-th water-in-oil droplet containing the liposome;
(f′) sequentially fusing the n-th water-in-oil droplet containing the continuously obtained source cell of the n−1-th stage with the continuously formed n+1-th water-in-oil droplet to continuously obtain an n+2-th water-in-oil droplet;
(g′) sequentially introducing the n−1-th active agent into the source cell of the n−1-th stage, in the continuously obtained n+2-th water-in-oil droplet, to continuously obtain the target cell of the n-th stage;
(h) mixing an n-th liposomal aqueous solution phase encapsulating an n-th active agent to be introduced into the source cell with the oil phase to continuously form an n+2-th water-in-oil droplet containing the liposome;
(i) sequentially fusing the n+1-th water-in-oil droplet containing the continuously obtained source cell of the n-th stage and the continuously formed n+2-th water-in-oil droplet to continuously obtain an n+3-th water-in-oil droplet; and
(j) sequentially introducing the n-th active agent into the source cell of the n-th stage, in the continuously obtained n+3-th water-in-oil droplet, to continuously obtain the source cell, wherein
the processes (e) to (j) are repeated n−1 times in the same manner, and the second to n-th active agents are sequentially introduced into the source cells of the second to n-th stages, respectively.
(5) The method of one of (1) to (4), wherein
the active agent is a gene to be introduced.
(6) The method of one of (1) to (5), wherein
fusing between the first water-in-oil droplet and the second water-in-oil droplet is executed at a constant rate, for example, 1:1 and/or at a constant rate.
(7) The method of one of (1) to (6), wherein
each of the liposomal aqueous solution phase and an aqueous solution phase containing the source cell contains a surfactant.
(8) The method of one of (1) to (7), wherein
a lipid membrane constituting the liposome contains FFT10 and/or FFT20 represented by the following chemical formula.
(9) The method of one of (1) to (8), wherein
the method is conducted in a microfluidic channel.
(10) The method of one of (1) to (9), wherein
the fusing includes applying of at least one type of external energy selected from electricity, temperature, magnetism, light, and ultrasonic wave to promote the fusing.
(11) The method of one of (1) to (10), wherein
the method includes adjusting at least one environmental condition selected from time, temperature, and gas concentration to promote introduction of an active agent into the cell.
(12) The method of one of (1) to (11), further comprising:
a system of conducting the recovery at a high efficiency.
(13) The method of one of (1) to (12), wherein
mixing the aqueous solution phase with the oil phase is conducted by merging microfluidic channels included in the respective phases, and the two types of water-in-oil droplets are brought into contact with each other by merging the microfluidic channels included in the phases.
(14) A microfluidic system for conducting a method of (1) to (13) in a continuous space.
(15) The system of (14), comprising a swirl channel for preparing the water-in-oil droplet.
(16) A microfluidic system for preparing a target cell by introducing an active agent into a source cell, the system comprising:
a first water-in-oil droplet forming channel comprising a first oil phase feed channel and a first aqueous solution phase feed channel joining the first oil phase feed channel, to prepare a first water-in-oil droplet containing a source cell suspension in an aqueous solution phase;
a second water-in-oil droplet forming channel comprising a second oil phase feed channel and a second aqueous solution phase feed channel joining the second oil phase feed channel, to prepare a second water-in-oil droplet containing a first liposome encapsulating a first active agent, in an aqueous solution phase;
a fusion part connected to merge the first water-in-oil droplet forming channel and the second water-in-oil droplet forming channel, to fuse the first water-in-oil droplet and the second water-in-oil droplet;
an introduction part connected to the fusion part, bringing the first liposome and the source cell into contact with each other and introducing the active agent into the source cell; and
an opening part discharging a target cell obtained by the introduction.
(17) A microfluidic system for preparing a target cell by introducing a first active agent and a second active agent into a source cell, the system comprising:
a first water-in-oil droplet forming channel comprising a first oil phase feed channel and a first aqueous solution phase feed channel joining the first oil phase feed channel, to prepare a first water-in-oil droplet containing a cell suspension containing a source cell of a first stage, in an aqueous solution
a second water-in-oil droplet forming channel comprising a second oil phase feed channel and a second aqueous solution phase feed channel joining the second oil phase feed channel, to prepare a second water-in-oil droplet containing a first liposome encapsulating a first active agent, in an aqueous solution phase;
a first fusion part connected to merge the first water-in-oil droplet forming channel and the second water-in-oil droplet forming channel, to fuse the first water-in-oil droplet and the second water-in-oil droplet and obtain a first fused water droplet;
a first active agent introduction part connected to the first fusion part, bringing the first liposome and the source cell into contact with each other, introducing the active agent into the source cell, and obtaining the source cell of a second stage;
a third water-in-oil droplet forming channel comprising a third oil phase feed channel and a third aqueous solution phase feed channel joining the third oil phase feed channel, to prepare a third water-in-oil droplet containing a second liposome encapsulating a second active agent, in an aqueous solution phase;
a second fusion part connected to merge the first active agent introduction part and the third water-in-oil droplet forming channel, to obtain a second fused water droplet for fusing the first fused water droplet and the third water-in-oil droplet;
an introduction part connected to the second fusion part, bringing the second liposome and the source cell of the second stage into contact with each other, and introducing the second active agent into the source cell of the second stage; and
an opening part discharging a target cell obtained by the introduction.
(18) A microfluidic system for preparing a target cell by introducing first to n-th active agents (where n is an integer larger than or equal to 3) into a source cell, the system comprising:
a first water-in-oil droplet forming channel comprising a first oil phase feed channel and a first aqueous solution phase feed channel joining the first oil phase feed channel, to prepare a first water-in-oil droplet containing a cell suspension containing a source cell of a first stage, in an aqueous solution phase;
a second water-in-oil droplet forming channel comprising a second oil phase feed channel and a second aqueous solution phase feed channel joining the second oil phase feed channel, to prepare a second water-in-oil droplet containing a first liposome encapsulating a first active agent, in an aqueous solution phase;
a first fusion part connected to merge the first water-in-oil droplet forming channel and the second water-in-oil droplet forming channel, to fuse the first water-in-oil droplet and the second water-in-oil droplet and obtain a first fused water droplet;
a first active agent introduction part connected to the first fusion part, bringing the first liposome and the source cell into contact with each other, introducing the active agent into the source cell, and obtaining the source cell of a second stage;
third to n+1-th water-in-oil droplet forming channels comprising third to n+1-th oil phase feed channels and third to n+1-th aqueous solution phase feed channels joining the third to n+1-th oil phase feed channels, respectively, to prepare third to n+1-th water-in-oil droplets containing second to n-th liposomes encapsulating second to n-th active agents, respectively, in an aqueous solution phase;
second to n-th fusion parts connected to merge the first to n−1-th active agent introduction parts and the third to n+1-th water-in-oil droplet forming channels, respectively, to obtain second to n-th fused water droplets for fusing the first to n−1-th fused water droplets and the third to n+1-th water-in-oil droplets;
an introduction part connected to the second to n-th fusion parts, bringing the second to n-th liposomes and the source cells of the second to n-th stages into contact with each other, and introducing the second to n-th active agents into the source cells of the second to n-th stages; and
an opening part discharging a target cell obtained by the introduction.
(19) The system of one of (14) to (18), further comprising:
a control part controlling a flow rate and/or a flow rate in the system.
(20) The system of one of (14) to (19), wherein
the fusing part and/or the introduction part comprises an electrical stimulation mechanism, a difference in shape from a channel before and after the mechanism, and a heating mechanism and/or a magnetic field generation mechanism.
(21) The system of one of (14) to (20), further comprising:
a timing control part controlling timing at which the water-in-oil droplet moves.
(22) The system of one of (14) to (21), further comprising:
a temperature management mechanism managing a temperature in the system.
(23) The system of one of (14) to (23), wherein
the fusing part and/or the introducing part comprises an annular channel and/or a swirl channel.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A target cell preparation method comprising introducing an active agent into a source cell, the method comprising:
forming a first water-in-oil droplet containing the source cell by mixing an aqueous solution containing the source cell with an oil phase;
forming a second water-in-oil droplet containing a liposome by mixing a liposomal aqueous solution encapsulating the active agent with an oil phase;
fusing the formed first water-in-oil droplet with the second water-in-oil droplet to form a fused water droplet; and
introducing the active agent into the source cells in the fused water droplet.

2. A target cell preparation method comprising introducing an active agent into a source cell, the method comprising:
(a) intermittently mixing a liposomal aqueous solution phase encapsulating an active agent to be introduced into the source cell, and an oil phase, to continuously form a first water-in-oil droplet containing the liposome;
(b) intermittently mixing the aqueous solution phase containing the source cell, and the oil phase, to continuously form a second water-in-oil droplet containing the source cell;
(c) sequentially fusing the successively formed first water-in-oil droplet, and the successively formed second water-in-oil droplet, to successively obtain third water-in-oil droplets; and
(d) introducing the active agent into the source cell in each of the continuously obtained third water-in-oil droplets, to continuously obtain the target cell.

3. A target cell preparation method comprising introducing a first active agent and a second active agent into a source cell, the method comprising:
(a′) mixing a first liposomal aqueous solution phase encapsulating the first active agent to be introduced into the source cell of a first stage with an oil phase at a constant ratio and at a constant rate to continuously form a first water-in-oil droplet containing the liposome;
(b′) mixing the aqueous solution phase containing the source cell with the oil phase at a constant ratio and at a constant rate to continuously form a second water-in-oil droplet containing the source cell;
(c′) sequentially fusing the continuously formed first water-in-oil droplet and the continuously formed second water-in-oil droplet to continuously obtain a third water-in-oil droplet;
(d′) sequentially introducing the active agent into the source cell, in the continuously obtained third water-in-oil droplet, to continuously obtain a second-stage source cell;
(e) mixing a second liposomal aqueous solution phase encapsulating the second active agent to be introduced into the source cell with the oil phase to continuously form a fourth water-in-oil droplet containing the liposome;
(f) successively fusing the third water-in-oil droplet containing the continuously obtained second-stage source cell with the continuously formed fourth water-in-oil droplet to continuously obtain fifth water-in-oil droplets; and
(g) introducing the second active agent into the second-stage source cell, in each of the continuously obtained fifth water-in-oil droplets, to continuously obtain the target cell.

4. A target cell preparation method comprising sequentially introducing all of first to n-th active agents (where n is an integer larger than or equal to 3) into a source cell, the method comprising:
(a″) mixing a first liposomal aqueous solution phase encapsulating the first active agent to be introduced into the source cell of a first stage with an oil phase at a constant ratio and at a constant rate to continuously form a first water-in-oil droplet containing the liposome;
(b″) mixing an aqueous solution phase containing the source cell of the first stage with the oil phase at a constant ratio and at a constant rate to continuously form a second water-in-oil droplet containing the source cell;
(c″) sequentially fusing the continuously formed first water-in-oil droplet and the continuously formed second water-in-oil droplet to continuously obtain a third water-in-oil droplet;
(d″) sequentially introducing the active agent into the source cell, in the continuously obtained third water-in-oil droplet, to continuously obtain the source cell of a second stage;
(e′) mixing an n−1-th liposomal aqueous solution phase encapsulating an n−1-th active agent to be introduced into the source cell with the oil phase at a constant ratio and at a constant rate to continuously form an n+1-th water-in-oil droplet containing the liposome;
(f′) sequentially fusing the n-th water-in-oil droplet containing the continuously obtained source cell of the n−1-th stage with the continuously formed n+1-th water-in-oil droplet to continuously obtain an n+2-th water-in-oil droplet;
(g′) sequentially introducing the n−1-th active agent into the source cell of the n−1-th stage, in the continuously obtained n+2-th water-in-oil droplet, to continuously obtain the target cell of the n-th stage;
(h) mixing an n-th liposomal aqueous solution phase encapsulating an n-th active agent to be introduced into the source cell with the oil phase at a constant ratio and at a constant rate to continuously form an n+2-th water-in-oil droplet containing the liposome;
(i) sequentially fusing the n+1-th water-in-oil droplet containing the continuously obtained source cell of the n-th stage and the continuously formed n+2-th water-in-oil droplet to continuously obtain an n+3-th water-in-oil droplet; and
(j) sequentially introducing the n-th active agent into the source cell of the n-th stage, in the continuously obtained n+3-th water-in-oil droplet, to continuously obtain the source cell, wherein
the processes (e) to (j) are repeated n−1 times in the same manner, and the second to n-th active agents are sequentially introduced into the source cells of the second to n-th stages, respectively.

5. The method of claim 1, wherein
the active agent is a gene.

6. The method of claim 1, wherein
fusing between the water-in-oil droplets is executed at 1:1.

7. The method of claim 1, wherein
the oil phase encapsulating the liposome aqueous solution phase and an aqueous solution phase containing the source cell contains a surfactant.

8. The method of claim 1, wherein
a lipid membrane constituting the liposome contains FFT10 and/or FFT20 represented by the following chemical formula:

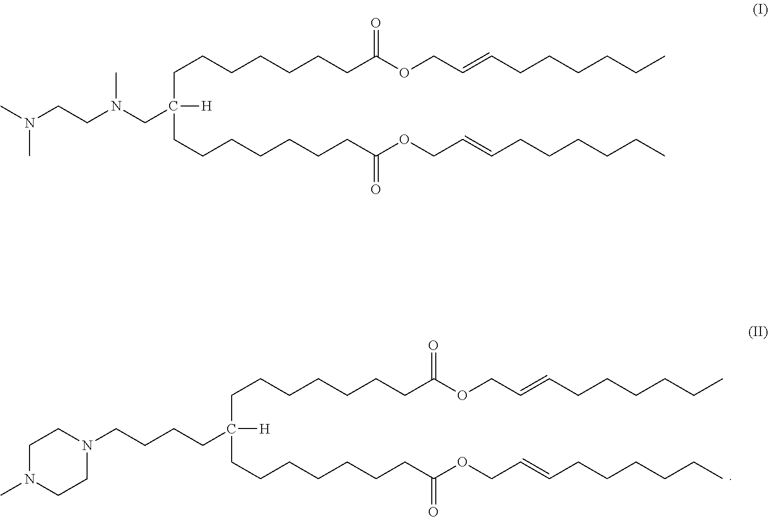

9. The method of claim 1, wherein
the method is conducted in a microfluidic channel.

10. The method of claim 1, wherein
mixing the aqueous solution phase and the oil phase and/or fusing water-in-oil droplets is conducted by joining contained microfluidic channels and/or a swirl channel obtained after the joining.

11. A microfluidic system for preparing a target cell by introducing an active agent into a source cell, the system comprising:
a first water-in-oil droplet forming channel comprising a first oil phase feed channel and a first aqueous solution phase feed channel joining the first oil phase feed channel, to prepare a first water-in-oil droplet containing a source cell suspension in an aqueous solution phase;
a second water-in-oil droplet forming channel comprising a second oil phase feed channel and a second aqueous solution phase feed channel joining the second oil phase feed channel, to prepare a second water-in-oil droplet containing a first liposome encapsulating a first active agent, in an aqueous solution phase;
a fusion part connected to merge the first water-in-oil droplet forming channel and the second water-in-oil droplet forming channel, to fuse the first water-in-oil droplet and the second water-in-oil droplet;
an introduction part connected to the fusion part, bringing the first liposome and the source cell into contact with each other and introducing the active agent into the source cell; and
an opening part discharging a target cell obtained by the introduction.

12. A microfluidic system for preparing a target cell by introducing a first active agent and a second active agent into a source cell, the system comprising:
a first water-in-oil droplet forming channel comprising a first oil phase feed channel and a first aqueous solution phase feed channel joining the first oil phase feed channel, to prepare a first water-in-oil droplet containing a cell suspension containing a source cell of a first stage, in an aqueous solution phase;
a second water-in-oil droplet forming channel comprising a second oil phase feed channel and a second aqueous solution phase feed channel joining the second oil phase feed channel, to prepare a second water-in-oil droplet containing a first liposome encapsulating a first active agent, in an aqueous solution phase;
a first fusion part connected to merge the first water-in-oil droplet forming channel and the second water-in-oil droplet forming channel, to fuse the first water-in-oil droplet and the second water-in-oil droplet and obtain a first fused water droplet;
a first active agent introduction part connected to the first fusion part, bringing the first liposome and the source cell into contact with each other, introducing the active agent into the source cell, and obtaining the source cell of a second stage;
a third water-in-oil droplet forming channel comprising a third oil phase feed channel and a third aqueous solution phase feed channel joining the third oil phase feed channel, to prepare a third water-in-oil droplet containing a second liposome encapsulating a second active agent, in an aqueous solution phase;
a second fusion part connected to merge the first active agent introduction part and the third water-in-oil droplet forming channel, to obtain a second fused water droplet for fusing the first fused water droplet and the third water-in-oil droplet;
an introduction part connected to the second fusion part, bringing the second liposome and the source cell of the second stage into contact with each other, and introducing the second active agent into the source cell of the second stage; and
an opening part discharging a target cell obtained by the introduction.

13. A microfluidic system for preparing a target cell by introducing first to n-th active agents (where n is an integer larger than or equal to 3) into a source cell, the system comprising:
a first water-in-oil droplet forming channel comprising a first oil phase feed channel and a first aqueous solution phase feed channel joining the first oil phase feed channel, to prepare a first water-in-oil droplet containing a cell suspension containing a source cell of a first stage, in an aqueous solution phase;
a second water-in-oil droplet forming channel comprising a second oil phase feed channel and a second aqueous solution phase feed channel joining the second oil phase feed channel, to prepare a second water-in-oil droplet containing a first liposome encapsulating a first active agent, in an aqueous solution phase;
a first fusion part connected to merge the first water-in-oil droplet forming channel and the second water-in-oil droplet forming channel, to fuse the first water-in-oil droplet and the second water-in-oil droplet and obtain a first fused water droplet;
a first active agent introduction part connected to the first fusion part, bringing the first liposome and the source cell into contact with each other, introducing the active agent into the source cell, and obtaining the source cell of a second stage;
third to n+1-th water-in-oil droplet forming channels comprising third to n+1-th oil phase feed channels and third to n+1-th aqueous solution phase feed channels joining the third to n+1-th oil phase feed channels, respectively, to prepare third to n+1-th water-in-oil droplets containing second to n-th liposomes encapsulating second to n-th active agents, respectively, in an aqueous solution phase;
second to n-th fusion parts connected to merge the first to n−1-th active agent introduction parts and the third to n+1-th water-in-oil droplet forming channels, respectively, to obtain second to n-th fused water droplets for fusing the first to n−1-th fused water droplets and the third to n+1-th water-in-oil droplets;
an introduction part connected to the second to n-th fusion parts, bringing the second to n-th liposomes and the source cells of the second to n-th stages into contact with each other, and introducing the second to n-th active agents into the source cells of the second to n-th stages; and
an opening part discharging a target cell obtained by the introduction.

14. The method of claim 11, further comprising:
a control part controlling a flow rate and/or a flow rate in the system.

15. The method of claim 11, wherein
the fusing part and/or the introduction part comprises an electrical stimulation mechanism, a difference in shape from a channel before and after the mechanism, and a heating mechanism and/or a magnetic field generation mechanism.

16. The method of claim 11, further comprising:
a timing control part controlling timing at which the water-in-oil droplet moves.

17. The method of claim 11, further comprising:
a temperature management mechanism managing a temperature in the system.

18. The method of claim 11, wherein
the fusing part and/or the introducing part comprises an annular channel and/or a swirl channel.
